SEQUENCE LISTING (17 sequences)

SEQ ID NO: 1 - protein, 8 aa, Artificial Sequence
  peptide
WKTIITKN

SEQ ID NO: 2 - protein, 12 aa, Artificial Sequence
  epitope
GSETWKTIITKN

SEQ ID NO: 3 - protein, 18 aa, Artificial Sequence
  epitope
IGSETWKTIITKNLHYKD

SEQ ID NO: 4 - protein, 13 aa, Artificial Sequence
  epitope
GSETWKTIITKNC

SEQ ID NO: 5 - protein, 14 aa, Artificial Sequence
  epitope
GGGSETWKTIITKN

SEQ ID NO: 6 - protein, 10 aa, Artificial Sequence
  CDR1-VH from monoclonal antibody 5.5.4
GYTFTDYVLH

SEQ ID NO: 7 - protein, 14 aa, Artificial Sequence
  CDR2-VH from monoclonal antibody 5.5.4
IDSSNIKYNENFRD

SEQ ID NO: 8 - protein, 8 aa, Artificial Sequence
  CDR3-VH from monoclonal antibody 5.5.4
GDYDPFDY

SEQ ID NO: 9 - protein, 11 aa, Artificial Sequence
  CDR1-VL from monoclonal antibody 5.5.4
KASQDISSYLS

SEQ ID NO: 10 - protein, 7 aa, Artificial Sequence
  CDR2-VL from monoclonal antibody 5.5.4
RANRLLD

SEQ ID NO: 11 - protein, 9 aa, Artificial Sequence
  CDR3-VL from monoclonal antibody 5.5.4
LQYDEFPLT

SEQ ID NO: 12 - protein, 10 aa, Artificial Sequence
  CDR1-VH from monoclonal antibody 4.4.2
GFNIEDTYMH

SEQ ID NO: 13 - protein, 17 aa, Artificial Sequence
  CDR2-VH from monoclonal antibody 4.4.2
RIDPANGNTKYDPKFRD

SEQ ID NO: 14 - protein, 11 aa, Artificial Sequence
  CDR3-VH from monoclonal antibody 4.4.2
SYGNYDWYFDV

SEQ ID NO: 15 - protein, 10 aa, Artificial Sequence
  CDR1-VL from monoclonal antibody 4.4.2
SASSSVSYMH

SEQ ID NO: 16 - protein, 7 aa, Artificial Sequence
  CDR2-VL from monoclonal antibody 4.4.2
DTSKLAS

SEQ ID NO: 17 - protein, 9 aa, Artificial Sequence
  CDR3-VL from monoclonal antibody 4.4.2
QQWSSHPLT

(19) United States
(12) Patent Application Publication
(10) Pub. No.: US 2024/0182589 A1
(43) Pub. Date: Jun. 6, 2024

(54) ANTIBODY AND USE THEREOF FOR THE TREATMENT OF CANCER

(71) Applicant: CONSEJO SUPERIOR DE INVESTIGACIONES CIENTÍFICAS, Madrid (ES)

(72) Inventors: José Ignacio CASAL ÁLVAREZ, Madrid (ES); Rubén Álvaro BARTOLOME CONDE, Madrid (ES); Marta JAÉN CASTAÑO, Madrid (ES)

(21) Appl. No.: 18/553,464

(22) PCT Filed: Mar. 30, 2022
(86) PCT No.: PCT/EP2022/058454

§ 371 Date: Sep. 29, 2023

(30) Foreign Application Priority Data
Mar. 31, 2021 (EP) 21382266.1

(51) Int. Cl. C07K 16/28; A61K 39/00; A61P 35/04; G01N 33/68

(52) U.S. Cl. CPC C07K 16/2866; A61P 35/04; G01N 33/6869; A61K 2039/505; C07K 2317/565; G01N 2333/7155; G01N 2800/52

(57) ABSTRACT

The present invention relates to a monoclonal antibody that specifically binds to an epitope comprising the amino acid sequence SEQ ID NO: 2, SEQ ID NO: 3, SEQ ID NO: 4 or SEQ ID NO: 5 and capable of blocking the binding of IL-13 to IL13Rα2 or inhibiting the IL13/IL13Rα2 signaling, as well as the antiserum, the kit and the pharmaceutical composition comprising said monoclonal antibody. The invention also relates to the use of the antibody, the antiserum and the pharmaceutical composition in the treatment of cancer, specifically, cancer metastasis.

The present invention relates to a monoclonal antibody that specifically binds to an epitope comprising the amino acid sequence SEQ ID NO: 2, SEQ ID NO: 3, SEQ ID NO: 4 or SEQ ID NO: 5 and capable of blocking the binding of IL-13 to IL13Rα2 or inhibiting the IL13/IL13Rα2 signaling, as well as to its use in the treatment of cancer characterized by the expression of IL13Rα2, specifically, cancer metastasis. Thus, the present invention relates to medical field, particularly, to the treatment of cancer.

### BACKGROUND ART

Colorectal cancer (CRC) is among the four most lethal tumors worldwide. Europe shows a high incidence, with the majority of its countries having a rate of 26 deaths per 100,000 people. Mortality rate is very high among patients with metastasis and about 50% of the patients present with metastasis at diagnosis. Current treatment of metastatic CRC includes combinations of anti-EGFR monoclonal antibodies (mAbs) (Cetuximab, Panitumumab) or anti-VEGF (Bevacizumab) with different FOLFOX and FOLFIRI regimes. EGFR-based therapies suffer from low efficacy in those patients that present activating mutations in KRAS, BRAF and alterations in PIK3CA, and may develop some side effects at different extent. In patients with KRAS and BRAF mutations there is a limited clinical benefit from this therapy and the overall survival improvement is rather modest. It has been estimated that near 80% of metastatic CRC patients do not benefit from anti-EGFR therapy. Other promising immunotherapies as PD-1 blockade are also quite ineffective in CRC. Therefore, patients will benefit from novel targets and novel strategies to develop therapeutic antibodies for a more successful treatment of disseminated colorectal cancer.

IL-13 receptor α2 (IL13Rα2) is a cancer/testis-like tumor antigen, overexpressed in multiple tumors such as CRC, renal cell carcinoma, pancreatic, melanoma, head and neck, mesothelioma, ovarian cancer and glioblastoma, among others. In CRC, higher expression was observed in T3 or T4 tumors as compared with T1 or T2. Initially considered a decoy receptor, we and others have demonstrated beyond reasonable doubt that IL13Rα2 is also a functional receptor for IL-13 signaling transmission in cancer cells. The binding of IL-13 to IL13Rα2 triggers STAT6-independent cellular pathways, through different mediators including the phosphatase PTP1B and the scaffold protein FAM120A. Indeed, the IL-13/IL13Rα2 signaling axis activates Src through PTP1B and then activates the Ras→Raf→MAPK cascade followed by the AP-1 transcriptional pathway in a number of human cancers. As observed for many other receptors, IL-13 binding may induce receptor dimerization and/or internalization, where IL13Rα2 associates with multiple intracellular traffic proteins, being its recycling tightly controlled to regulate the surface expression levels and the amount of free receptor on the surface.

Due to its expression in advanced stages of cancer, IL13Rα2 has been postulated as a target for cancer therapy through multiple approaches. IL13Rα2 presents multiple advantages as therapeutic target; it is a signaling receptor specific of metastatic cancer cells, as it is only expressed in testis cells and some immune cells in adults. IL13Rα2 does not present a significant mutation rate in colorectal cancer avoiding extensive testing on natural variants. Blocking the IL-13/IL13Rα2 signaling capacity in metastatic cells should facilitate the development of new therapeutic strategies. Indeed, an IL13Rα2 D1 peptide (GSETWKTIITKN, SEQ ID NO: 2) located at the IL-13 binding site showed strong inhibition capacity for the IL-13/IL13Rα2 signaling cascade in metastatic CRC and glioblastoma. This 12 amino acid-long D1 peptide contains a lineal sequence (81-WKTIITKN-88 (SEQ ID NO: 1)) from IL13Rα2 that is highly preserved in many mammalian species.

Thus, IL13Rα2 is a promising therapeutic target in different cancers. Still, no specific antagonists have reached the clinics yet. Therefore, the development of IL13Rα2-specific performing clinical agents remains of interest.

### SUMMARY OF THE INVENTION

By means of a functional screening based on inhibition of cellular invasion assays, the inventors of the present invention have developed a monoclonal antibody (mAb) which, surprisingly, inhibits IL-13 promoted adhesion, migration and, particularly, invasive capacity of differentiated colorectal cancer metastatic cells, in a more effective way than other antibodies of the state of the art. This inhibition is triggered by blocking the ligand-binding receptor activation that includes Src and AKT phosphorylation, among other downstream receptor signaling mediators. The blocking effectivity is related to the IL13Rα2 (interleukin 13 receptor α2) expression level, which appears to correlate with the differentiation grade of the metastatic cell lines. Despite this mAb partially blocked IL-13 mediated receptor internalization from the cancer cell surface it still promotes receptor degradation. Compared with other IL13Rα2-specific antibodies, the antibody of the present invention exhibits a superior efficacy to inhibit metastatic growth in vivo, providing a complete mouse survival in different conditions, including established metastasis.

In view of the foregoing, in a first aspect the present invention relates to a monoclonal antibody that specifically binds to an epitope comprising, or consisting of, the amino acid sequence GSETWKTIITKN (SEQ ID NO: 2), IGSETWKTIITKNLHYKD (SEQ ID NO: 3), GSETWKTIITKNC (SEQ ID NO: 4) or GGGSETWKTIITKN (SEQ ID NO: 5), and capable of blocking the binding of IL-13 to IL13Rα2 or inhibiting the IL13/IL13Rα2 signaling (hereinafter “monoclonal antibody of the invention”).

The term “antibody”, “antibodies”, “ab”, “Ab” or “immunoglobulin” are used interchangeably in the broadest sense as used in the present invention, and relates to immunoglobulin molecules and immunologically active portions (or fragments) of immunoglobulin molecules (also called “antigen binding fragments”). That is, it refers to molecules that specifically bind (are immunoreactive) to an antigen, such as, for example, a peptide or a protein (an immunogen or epitope). The term “epitope” is a region of an antigen that is bound by an antigen binding protein, including antibodies. In the present invention, the epitope is located in the IL13Rα2 protein. The term “antibody” comprises monoclonal antibodies and polyclonal antibodies, and in the present invention the antibody is monoclonal, and it refers to an antibody that is intact or to immunologically active fragments of the same, and includes human, humanized and non-human, recombinant, chimeric and synthetic antibodies. In the context of this invention, the term antibody refers to the immunoglobulin that the animal or a hybrid cell has specifically synthesized against the sequence described in the first aspect of the present invention.

The term “antibody fragment”, as used herein, refers to a fragment of an antibody such as, for example, Fv, Fab, F(ab′)2, and Fab′ fragments. Various techniques have been developed for the production of antibody fragments. Traditionally, these fragments were derived via proteolytic digestion of intact antibodies but more recently these fragments can be produced directly by recombinant host cells. Papain digestion of antibodies produces two identical antigen-binding fragments, called “Fab” fragments, each with a single antigen-binding site, and a residual “Fc” fragment, which name reflects its ability to crystallize readily. Pepsin treatment yields an F(ab′)₂ fragment that has two antigen-binding sites and is still capable of cross-linking antigen. “Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind the antigen, although with lower affinity than the entire binding site. The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region.

In a particular embodiment, the antigen-binding fragment is selected from the group consisting of Fv, Fab, F(ab′)₂, and Fab′.

As use herein, “Monoclonal antibodies” (or mAbs) are homogenous populations of identical antibodies, produced by a hybridoma, that is, a hybrid cell that is the product of the fusion of a clone of B lymphocytes descendant of a single and unique stem cell and a plasma cell tumor, which are directed against a specific site or antigenic determinant. The method for obtaining monoclonal antibodies of the invention can be carried out according to conventional methods known in the state of the art. Optionally, said antibodies can be purified by conventional means, such as affinity chromatography, protein A-Sepharose, hydroxyapatite chromatography, gel electrophoresis or dialysis.

As is known by a person skilled in the art, there are five isotypes or main classes of immunoglobulins: immunoglobulin M (IgM), immunoglobulin D (IgD), immunoglobulin G (lgG) (which in turn have the following subtypes in mice: IgG1, IgG2a, IgG2b and IgG3), immunoglobulin A (IgA) and immunoglobulin E (IgE).

The mAb of the invention specifically binds to an epitope comprising, or consisting of, the amino acid sequence SEQ ID NO: 1. In a particular embodiment of the mAb of the invention, the epitope comprises, or consist of the amino acid sequence

(SEQ ID NO: 2)
GSETWKTIITKN,
(SEQ ID NO: 3)
IGSETWKTIITKNLHYKD,
(SEQ ID NO: 4)
GSETWKTIITKNC
or
(SEQ ID NO: 5)
GGGSETWKTIITKN

As used herein, the term “specific recognition” or “specific binding” is understood as the binding (reaction, interaction or specific binding) between the antibody of the invention and the epitope comprising, or consisting of, the sequences SEQ ID NO:2, SEQ ID NO: 3, SEQ ID NO: 4 or SEQ ID NO: 5. By “binding”, “binds”, or the like, it is intended that the antibody, or any antigen binding fragment thereof, forms a complex with an antigen that is relatively stable under physiologic conditions. Methods for determining whether two molecules bind are well known in the art and include, for example, equilibrium dialysis, surface plasmon resonance, and the like. For the avoidance of doubt, it does not mean that the said antibody could not bind or interfere, at a low level, to another antigen.

A technical feature of the monoclonal antibody of the invention is its capacity of inhibiting the IL-13/IL13Rα2-mediated signaling. As used herein, the expression “inhibiting the IL-13/IL13Rα2-mediated signaling” means that the binding of IL-13 to IL13Rα2 is blocked resulting in that the signal induced by IL-13 and mediated by IL13Rα2 through Scr is inhibited. Thus, the expressions “inhibiting the IL-13/IL13Rα2-mediated signaling” and “blocking the binding of IL-13 to IL13Rα2” are equivalent in the present invention. The ability of an antibody to block the binding of IL-13 to IL13Rα2, or to inhibit the IL13/IL13Rα2 signaling, can be determined by measuring the ability of the antibody to block the activation of IL-13 targets mediated by IL13Rα2, namely, FAK (focal adhesion kinase), ERK1/2 (extracellular signal-regulated kinase 1/2), Src (proto-oncogene tyrosine kinase Src) and AKT (RAC-alpha serine/threonine-protein kinase). The activation of FAK, ERK1/2, Src and AKT by IL-13 can be determined by any suitable method known by the skilled person, for 10 example, by determining the amount of the corresponding phosphorylated forms phospho-FAK, phospho-ERK1/2, phospho-Src and phospho-AKT after IL-13 treatment, as described in the examples of the present document.

As explained above, the present invention also encompasses immunologically active portions (or fragments) of immunoglobulin molecules, also called herein “antigen binding fragments”. Examples of “antigen binding fragments” are selected from the group consisting of Fv, scFv (sc for single chain), Fab, F(ab′)2, Fab′, scFv-Fc fragments or diabodies, or any fragment of which the half-life time would have been increased by chemical modification, such as the addition of poly(alkylene) glycol such as poly(ethylene) glycol (“PEGylation”) (pegylated fragments called Fv-PEG, scFv-PEG, Fab-PEG, F(ab′)2-PEG or Fab′-PEG) (“PEG” for Poly(Ethylene) Glycol), or by incorporation into a liposome, wherein said fragments specifically bind to an epitope comprising, or consisting of, the amino acid sequences SEQ ID NO: 2, SEQ ID NO: 3, SEQ ID NO: 4 or SEQ ID NO: 5, and are capable of blocking the binding of IL-13 to IL13Rα2. The “antigen binding fragments” will comprise a partial sequence of the heavy or light variable chain of the antibody from which they are derived, said partial sequence being sufficient to retain the same specificity of binding as the antibody from which it is descended and a sufficient affinity, preferably at least equal to 1/100, in a more preferred manner to at least 1/10, of the affinity of the antibody from which it is descended, with respect to the target.

Any mAb that specifically binds to an epitope comprising, or consisting of, the amino acid sequence SEQ ID NO: 2, SEQ ID NO: 3, SEQ ID NO: 4 or SEQ ID NO: 5, and capable of blocking the binding of IL-13 to IL13Rα2 or of inhibiting the IL13/IL13Rα2 signaling falls within the context of the present invention. Nevertheless, in particular embodiment, the mAb of the invention is selected from the group consisting of
a) an antibody (also called 5.5.4 herein) comprising
   the three heavy-chain CDRs of sequences:

(SEQ ID NO: 6)
CDR1-VH: GYTFTDYVLH,
(SEQ ID NO: 7)
CDR2-VH: IDSSNIKYNENFRD,
and
(SEQ ID NO: 8)
CDR3-VH: GDYDPFDY;

or a functionally equivalent variant of said CDRs and
the three light-chain CDRs of sequences:

(SEQ ID NO: 9)
CDR1-VL: KASQDISSYLS
(SEQ ID NO: 10)
CDR2-VL: RANRLLD,
and
(SEQ ID NO: 11)
CDR3-VL: LQYDEFPLT;

or a functionally equivalent variant of said CDRs and
b) an antibody (also called 4.4.2 herein) comprising
   the three heavy-chain CDRs of sequences:

(SEQ ID NO: 12)
CDR1-VH: GFNIEDTYMH
(SEQ ID NO: 13)
CDR2-VH: RIDPANGNTKYDPKFRD,
and
(SEQ ID NO: 14)
CDR3-VH: SYGNYDWYFDV,

or a functionally equivalent variant of said CDRs and
the three light-chain CDRs of sequences:

(SEQ ID NO: 15)
CDR1-VL: SASSSVSYMH
(SEQ ID NO: 16)
CDR2-VL: DTSKLAS,
and
(SEQ ID NO: 17)
CDR3-VL: QQWSSHPLT

or a functionally equivalent variant of said CDRs.

In a particular embodiment, the “antigen binding fragments” of the mAb 5.5.4 or 4.4.2 will comprise at least the three CDRs CDR-H1, CDR-H2 and CDR-H3 of the heavy variable chain and the three CDRs CDR-L1, CDR-L2 and CDR-L3 of the light variable chain of the antibody from which they are derived.

As it is used herein, the term “functionally equivalent variant of a CDR sequence” refers to a sequence variant of a particular CDR sequence having substantially similar sequence identity with it and substantially maintaining its capacity to bind to its cognate antigen and/or the capacity to inhibit IL13/IL13Rα signalling when being part of an antibody or antibody fragment as the ones described herein. For example, a functionally equivalent variant of a CDR sequence may be a polypeptide sequence derivative of said sequence comprising the addition, deletion or substitution of one or more amino acids.

Functionally equivalent variants of a CDR sequence according to the invention include CDR sequences having at least approximately 70%, at least 75%, at least 80%, at least 85%, at least 90%, at least 91%, at least 92%, at least 93%, at least 94%, at least 95%, at least 96%, at least 97%, at least 98% or at least 99% sequence identity with the corresponding amino acid sequences shown in one of SEQ ID NOs: 6 to 17. It is also contemplated that functionally equivalent variants of a CDR sequence comprise additions consisting of at least 1 amino acid, or at least 2 amino acids, or at least 3 amino acids, or at least 4 amino acids, or at least 5 amino acids, or at least 6 amino acids, or at least 7 amino acids, or at least 8 amino acids, or at least 9 amino acids, or at least 10 amino acids or more amino acids at the N-terminus, or at the C-terminus, or both at the N- and C-terminus of the corresponding amino acid sequence shown in one of SEQ ID NOs: 6-17. Likewise, it is also contemplated that variants comprise deletions consisting of at least 1 amino acid, or at least 2 amino acids, or at least 3 amino acids, or at least 4 amino acids, or at least 5 amino acids, or at least 6 amino acids, or at least 7 amino acids, or at least 8 amino acids, or at least 9 amino acids, or at least 10 amino acids or more amino acids at the N-terminus, or at the C-terminus, or both at the N- and C-terminus of the corresponding amino acid sequence shown in one of SEQ ID NOs: 6-17.

Functionally equivalent variants a CDR sequence according to the invention will preferably maintain at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 100%, at least 105%, at least 110%, at least 115%, at least 120%, at least 125%, at least 130%, at least 135%, at least 140%, at least 145%, at least 150%, at least 200% or more of the capacity of the corresponding amino acid sequence shown in one of SEQ ID NOs: 6-17 to bind to its cognate antigen when being part of an antibody or antibody fragment as the ones of the invention. This capacity to bind to its cognate antigen may be determined as a value of affinity, avidity, specificity and/or selectivity of the antibody or antibody fragment to its cognate antigen.

The capacity of the antibody and antigen-binding fragments according to the invention, and in particular of the antibody or antibody fragment as described herein, to bind to an epitope comprising, or consisting of, the amino acid sequence SEQ ID NO: 2, SEQ ID NO: 3, SEQ ID NO: 4 or SEQ ID NO: 5, can be determined by a number of assays that are well known in the art. Preferably, the binding capacity of the binding agents is determined by immunoprecipitation or by an in vitro binding assay, such as radioimmunoassay (RIA), enzyme-linked immunoabsorbent assay (ELISA), surface plasmon resonance or by immunofluorescent techniques such as immunohistochemistry (IHC), fluorescence microscopy or flow cytometry.

The capacity of the antibody and antigen-binding fragments according to the invention to inhibit IL13/IL13Rα signalling can be determined by a number of assays that are well-known in the art, for example, competitions assays between the antibody or antigen-binding fragment and the recombinant IL-13 for the binding to the IL13Rα2 receptor or assays to detect inhibition of the SCR activation pathway, as described in the Examples herein provided.

In another aspect, the present invention relates to a chimeric antigen receptor comprising the antibody or antigen-binding fragment of the first aspect. The term “chimeric antigen receptor” or “CAR”, also known as “T-body, artificial T cell receptors” and “chimeric immune receptors” (CIR), refers to an engineered receptor, which graft an arbitrary specificity onto an immune effector cell. In a classical CAR, the specificity of a monoclonal antibody is grafted onto an immune cell, for example, a T cell. CARs are therefore fusion proteins which comprise at least, an extracellular domain or antigen binding domain capable of binding to an antigen, a transmembrane domain derived from a polypeptide different from a polypeptide from which the extracellular domain is derived, and at least one intracellular costimulatory domain.

In another aspect, the present invention relates to an immune cell comprising a chimeric antigen receptor comprising the antibody or antigen-binding fragment of the first aspect. In a particular embodiment the immune cell is selected from the group consisting of is a T cell (CAR-T cell), or a NK cell (CAR-NK) or a macrophage.

In second aspect, the present invention relates to an isolated nucleic acid coding for a monoclonal antibody, or for an antigen binding fragment thereof, according to the present invention. Isolated nucleic acids obtained by recombinant genetics, by means, for example, of host cells, or obtained by chemical synthesis should also be mentioned here. The invention also relates to a vector comprising a nucleic acid coding for an antibody, or for an antigen binding fragment thereof, according to the invention. The vectors are, for example, vectors of plasmid or viral origin. They are used to transform host cells in order to clone or express the nucleotide sequences of the invention. The invention also relates to isolated host cells transformed by or comprising a vector as above described. The host cell can be selected among prokaryotic or eukaryotic systems such as bacterial cells, for example, but also yeast cells or animal cells, notably mammal cells (with the exception of human). Insect or plant cells can also be used. The invention also relates to animals, other than human, that have a transformed cell.

In a third aspect, the present invention relates to an antiserum, hereinafter “the antiserum of the invention”, comprising the monoclonal antibody or antigen-binding fragment of the invention.

In the present description, the term “antiserum” relates to a serum obtained after the immunization of an animal with an immunogen. The antiserum comprises specific antibodies of said immunogen generated after the immune response produced in the animal. In the context of the present invention, the immunogen is the peptide comprising, or consisting of, the sequence SEQ ID NO: 2, SEQ ID NO: 3, SEQ ID NO: 4 or SEQ ID NO: 5. Thus, the antiserum of the invention comprises the peptide comprising, or consisting of, the sequence, SEQ ID NO: 2, SEQ ID NO: 3, SEQ ID NO: 4 or SEQ ID NO: 5, and the specific monoclonal antibodies generated against said sequence.

In a fourth aspect, the present invention relates to a cell, hereinafter “cell of the invention”, expressing the monoclonal antibody or antigen-binding fragment of the invention (hybridoma).

In a fifth aspect, the present invention relates to a pharmaceutical composition comprising the monoclonal antibody or antigen-binding fragment of the invention, or the antiserum of the invention, hereinafter, “pharmaceutical composition of the invention”.

As used in the present description, the term “pharmaceutical composition” refers to any substance used for the diagnosis, prevention, alleviation, treatment or cure of a disease in a human being or in animals. The pharmaceutical composition of the invention can be used alone or in combination with other pharmaceutical compositions.

In a particular embodiment, the pharmaceutical composition of the invention further comprises a pharmaceutically acceptable carrier or excipient.

The term “pharmaceutically acceptable excipient” refers to a substance which helps the absorption of the pharmaceutical composition comprising the monoclonal antibody of the invention, stabilizes said pharmaceutical composition or helps in the manufacture thereof in the sense of giving it consistency, form, flavor or any other specific functional characteristic. Thus, excipients could have the function of keeping the ingredients bound together, such as for example starches, sugars or celluloses, a sweetening function, a colorant function, a protection function, such as for example isolating it from the air and/or moisture, a filler function for a tablet, capsule or any other form of formulation, such as for example dibasic calcium phosphate, a disintegrating function to facilitate the dissolution of the components and their absorption, without excluding other types of excipients not mentioned in this paragraph.

A “pharmaceutically acceptable carrier” (or “pharmacologically acceptable”) refers to any substance, or combination of substances, known in the pharmaceutical sector, used in the manufacture of pharmaceutical forms of administration and includes, but is not limited to, solids, liquids, solvents or surfactants. The carrier can be an inert substance or have a similar action to any of the compounds of the present invention, having the function of facilitating the incorporation of the drug as well as other compounds, allowing for an improved dosage and administration or providing consistency and form to the pharmaceutical composition. When the dosage form is liquid, the carrier is the diluent. The term “pharmacologically acceptable” refers to the fact that the compound referred to is allowed and evaluated so that it does not cause harm to the organisms to which it is administered.

The pharmaceutical composition of the invention can be administered through any route of administration, and as such, said composition shall be formulated in the pharmaceutical form suitable to the chosen route of administration. Thus, the pharmaceutical composition of the invention can be administered by oral, nasal, ocular, topical, intradermic, intracranial or intravenous route. In a particular intravenous or the intraperitoneal embodiment, the route of administration of said is theintravenous or the intraperitoneal.

“Intravenous route” administration of the composition by the injection into the blood flow.

“Intraperitoneal route” is understood as the administration of the pharmaceutical composition by the injection into the peritoneum.

The pharmaceutical composition may further comprise anotheranother compound useful in the treatment of cancer. illustrative non-limitative examples of compoundscompounds useful in the treatment of cancer include mechlorethamine, chlorambucol, melphalen, chlorydrate, pipobromen, prednimustin, disodic-phosphate, estramustine, cyclophosphamide, altretamine, trofosfamide, sulfofosfamide, ifosfamide, thiotepa, triethylenamine, altetramine, carmustine, streptozocin, fotemustin, lomustine, busulfan, treosulfan, improsulfan, dacarbazine, cis-platinum, oxaliplatin, lobaplatin, heptaplatin, miriplatin hydrate, carboplatin, methotrexate, pemetrexed, 5-fluoruracil, floxuridine, 5-fluorodeoxyuridine, capecitabine, cytarabine, fludarabine, cytosine arabinoside, 6-mercaptopurine (6-MP), nelarabine, 6-thioguanine (6-TG), chlorodesoxyadenosine, 5-azacytidine, gemcitabine, cladribine, deoxycoformycin, tegafur, pentostatin, doxorubicin, daunorubicin, idarubicin, valrubicin, mitoxantrone, dactinomycin, mithramycin, plicamycin, mitomycin C, bleomycin, procarbazine, paclitaxel, docetaxel, vinblastine, vincristine, vindesine, vinorelbine, topotecan, irinotecan, etoposide, valrubicin, amrubicin hydrochloride, pirarubicin, elliptinium acetate, zorubicin, epirubicin, idarubicin and teniposide, razoxin, marimastat, batimastat, prinomastat, tanomastat, ilomastat, CGS-27023A, halofuginon, COL-3, neovastat, thalidomide, CDC 501, DMXAA, L-651582, squalamine, endostatin, SU5416, SU6668, interferon-alpha, EMD121974, interleukin-12, IM862, angiostatin, tamoxifen, toremifene, raloxifene, droloxifene, iodoxyfene, anastrozole, letrozole, exemestane, flutamide, nilutamide, sprironolactone, cyproterone acetate, finasteride, cimitidine, bortezomid, Velcade, bicalutamide, cyproterone, flutamide, fulvestran, exemestane, dasatinib, erlotinib, gefitinib, imatinib, lapatinib, nilotinib, sorafenib, sunitinib, retinoid, rexinoid, methoxsalene, methylaminolevulinate, aldesleukine, OCT-43, denileukin diflitox, interleukin-2, tasonermine, lentinan, sizofilan, roquinimex, pidotimod, pegademase, thymopentine, poly I:C, procodazol, Tic BCG, Corynebacterium parvum, NOV-002, ukrain, levamisole, 1311-chTNT, H-101, celmoleukin, interferon alfa2a, interferon alfa2b, interferon gammala, interleukin-2, mobenakin, Rexin-G, teceleukin, aclarubicin, actinomycin, arglabin, asparaginase, carzinophilin, chromomycin, daunomycin, leucovorin, masoprocol, neocarzinostatin, peplomycin, sarkomycin, solamargine, trabectedin, streptozocin, testosterone, kunecatechins, sinecatechins, alitretinoin, belotecan hydrocholoride, calusterone, dromostanolone, elliptinium acetate, ethinyl estradiol, etoposide, fluoxymesterone, formestane, fosfetrol, goserelin acetate, hexyl aminolevulinate, histrelin, hydroxyprogesterone, ixabepilone, leuprolide, medroxyprogesterone acetate, megesterol acetate, methylprednisolone, methyltestosterone, miltefosine, mitobronitol, nadrolone phenylpropionate, norethindrone acetate, prednisolone, prednisone, temsirrolimus, testolactone, triamconolone, triptorelin, vapreotide acetate, zinostatin stimalamer, amsacrine, arsenic trioxide, bisantrene hydrochloride, chlorambucil, chlortrianisene, cis-diamminedichloroplatinium, cyclophosphamide, diethylstilbestrol, hexamethylmelamine, hydroxyurea, lenalidomide, lonidamine, mechlorethanamine, mitotane, nedaplatin, nimustine hydrochloride, pamidronate, pipobroman, porfimer sodium, ranimustine, razoxane, semustine, sobuzoxane, mesylate, triethylenemelamine, zoledronic acid, camostat mesylate, fadrozole HCl, nafoxidine, aminoglutethimide, carmofur, clofarabine, cytosine arabinoside, decitabine, doxifluridine, enocitabine, fludarabne phosphate, fluorouracil, ftorafur, uracil mustard, abarelix, bexarotene, raltiterxed, tamibarotene, temozolomide, vorinostat, megastrol, clodronate disodium, levamisole, ferumoxytol, iron isomaltoside, celecoxib, ibudilast, bendamustine, altretamine, mitolactol, temsirolimus, pralatrexate, TS-1, decitabine, bicalutamide, flutamide, letrozole, clodronate disodium, degarelix, toremifene citrate, histamine dihydrochloride, DW-166HC, nitracrine, decitabine, irinoteacn hydrochloride, amsacrine, romidepsin, tretinoin, cabazitaxel, vandetanib, lenalidomide, ibandronic acid, miltefosine, vitespen, mifamurtide, nadroparin, granisetron, ondansetron, tropisetron, alizapride, ramosetron, dolasetron mesilate, fosaprepitant dimeglumine, nabilone, aprepitant, dronabinol, TY-10721, lisuride hydrogen maleate, epiceram, defibrotide, dabigatran etexilate, filgrastim, pegfilgrastim, reditux, epoetin, molgramostim, oprelvekin, sipuleucel-T, M-Vax, acetyl L-carnitine, donepezil hydrochloride, 5-aminolevulinic acid, methyl aminolevulinate, cetrorelix acetate, icodextrin, leuprorelin, metbylphenidate, octreotide, amlexanox, plerixafor, menatetrenone, anethole dithiolethione, doxercalciferol, cinacalcet hydrochloride, alefacept, romiplostim, thymoglobulin, thymalfasin, ubenimex, imiquimod, everolimus, sirolimus, H-101, lasofoxifene, trilostane, incadronate, gangliosides, pegaptanib octasodium, vertoporfin, minodronic acid, zoledronic acid, gallium nitrate, alendronate sodium, etidronate disodium, disodium pamidronate, dutasteride, sodium stibogluconate, armodafinil, dexrazoxane, amifostine, WF-10, temoporfin, darbepoetin alfa, ancestim, sargramostim, palifermin, R-744, nepidermin, oprelvekin, denileukin diftitox, crisantaspase, buserelin, deslorelin, lanreotide, octreotide, pilocarpine, bosentan, calicheamicin, maytansinoids, ciclonicate and pyrrolobenzodiazepines.

The pharmaceutical composition may include a single composition or separate compositions. For example, the antibody, or the antigen-binding fragment thereof, of the pharmaceutical composition may be a composition in parenteral dosage form, and the anti-cancer drug may be a composition in oral dosage form.

The pharmaceutical composition may include an effective amount of the antibody, or the antigen binding fragment thereof, alone or in combination with a cytotoxic agent. The term “effective amount” used herein refers to an amount sufficient to block the binding of IL-13 to IL13Rα2 and to prevent or treat cancer, in particular, cancer metastasis, in an individual who need such prevention or treatment. The effective amount may be appropriately selected depending on a selected cell or individual by one of ordinary skill in the art. For example, the effective amount may be determined depending on disease severity, a patient's age, body weight, health conditions, gender, a patient's drug sensitivity, administration duration, administration route, excretion rate, treatment duration, and other factors, including use of a drug in combination with or at the same time as the pharmaceutical composition, and other factors known in the medical field. The effective amount may be about 0.5 μg to about 2 g, about 1 μg to about 1 g, about 10 μg to about 500 mg, about 100 μg to about 100 mg, or about 1 mg to about 50 mg of the pharmaceutical composition.

A dose of the pharmaceutical composition may be, for example, about 0.001 mg/kg to about 100 mg/kg, about 0.01 mg/kg to about 10 mg/kg, or about 0.1 mg/kg to about 1 mg/kg when administered to an adult. The number of administrations may be, for example, once or multiple times a day, once a week, once in two weeks, once in three weeks, once in four weeks, or once a year.

In a sixth aspect, the present invention relates to a kit and/or device, hereinafter “kit and/or device of the invention”, comprising the monoclonal antibody or antigen-binding fragment, the antiserum, the pharmaceutical composition of the invention, and/or any combination thereof.

The kit and/or device of the invention can further comprise, but is not limited to, probes, buffers, enzymes, agents for preventing contamination, etc. On the other hand, the kit can include all of the necessary supports and containers for the start-up and optimization thereof. The kit can further contain other proteins, including antibodies or antigens, which serve as positive and negative controls. Preferably, this kit further comprises the instructions for detecting the epitope comprising, or consisting of, the sequence SEQ ID NO: 1, SEQ ID NO: 2, SEQ ID NO: 3, SEQ ID NO: 4 or SEQ ID NO: 5, preferably by means of an immunohistochemical assay, more preferably by means of ELISA, Western blot or immunofluorescence.

In a seventh aspect, the present invention relates to the monoclonal antibody or antigen-binding fragment, the immune cell comprising the chimeric antigen receptor, the antiserum, or the pharmaceutical composition of the invention, for use as a medicament. Alternatively, the present invention relates to the use of the monoclonal antibody or antigen-binding fragment, the immune cell comprising the chimeric antigen receptor, the antiserum, or the pharmaceutical composition of the invention, in the manufacture of a medicament. Hereinafter, “first medical use of the invention”.

In a particular embodiment of the seventh aspect of the invention, the monoclonal antibody is selected from the group consisting of
a) an antibody (also called 5.5.4 herein) comprising
   the three heavy-chain CDRs of sequences:

(SEQ ID NO: 6)
CDR1-VH: GYTFTDYVLH,
(SEQ ID NO: 7)
CDR2-VH: IDSSNIKYNENFRD,
and
(SEQ ID NO: 8)
CDR3-VH: GDYDPFDY

or a functionally equivalent variant of said CDRs; and
the three light-chain CDRs of sequences:

(SEQ ID NO: 9)
CDR1-VL: KASQDISSYLS
(SEQ ID NO: 10)
CDR2-VL: RANRLLD,
and
(SEQ ID NO: 11)
CDR3-VL: LQYDEFPLT

or a functionally equivalent variant of said CDRs; and
b) an antibody (also called 4.4.2 herein) comprising
   the three heavy-chain CDRs of sequences:

(SEQ ID NO: 12)
CDR1-VH: GFNIEDTYMH
(SEQ ID NO: 13)
CDR2-VH: RIDPANGNTKYDPKFRD,
and
(SEQ ID NO: 14)
CDR3-VH: SYGNYDWYFDV,

or a functionally equivalent variant of said CDRs and
the three light-chain CDRs of sequences:

(SEQ ID NO: 15)
CDR1-VL: SASSSVSYMH
(SEQ ID NO: 16)
CDR2-VL: DTSKLAS,
and
(SEQ ID NO: 17)
CDR3-VL: QQWSSHPLT

or a functionally equivalent variant of said CDRs.

In an even more particular embodiment of the seventh aspect of the invention, the monoclonal antibody comprises
the three heavy-chain CDRs of sequences:

(SEQ ID NO: 6)
CDR1-VH: GYTFTDYVLH,
(SEQ ID NO: 7)
CDR2-VH: IDSSNIKYNENFRD,
and
(SEQ ID NO: 8)
CDR3-VH: GDYDPFDY

or a functionally equivalent variant of said CDRs; and
the three light-chain CDRs of sequences:

(SEQ ID NO: 9)
CDR1-VL: KASQDISSYLS
(SEQ ID NO: 10)
CDR2-VL: RANRLLD,
and
(SEQ ID NO: 11)
CDR3-VL: LQYDEFPLT

or a functionally equivalent variant of said CDRs.

In an eighth aspect, the present invention relates to the monoclonal antibody or antigen-binding fragment, the immune cell comprising the chimeric antigen receptor, the antiserum, or the pharmaceutical composition of the invention, for use in the treatment of cancer in a subject, wherein the cancer is characterized by expressing IL13Rα2. Alternatively, the present invention relates to the use of the monoclonal antibody or antigen-binding fragment, the immune cell comprising the chimeric antigen receptor, the antiserum, or the pharmaceutical composition of the invention, in the manufacture of a medicament for the treatment of cancer in a subject, wherein the cancer is characterized by expressing IL13Rα2. Hereinafter, “second medical use of the invention”.

The mAb, the antiserum and the pharmaceutical composition of the invention have been defined previously together with their particular embodiments.

In a particular embodiment of the eighth aspect of the invention, the monoclonal antibody is selected from the group consisting of
a) an antibody (also called 5.5.4 herein) comprising
   the three heavy-chain CDRs of sequences:

(SEQ ID NO: 6)
CDR1-VH: GYTFTDYVLH,
(SEQ ID NO: 7)
CDR2-VH: IDSSNIKYNENFRD,
and
(SEQ ID NO: 8)
CDR3-VH: GDYDPFDY

or a functionally equivalent variant of said CDRs; and
the three light-chain CDRs of sequences:

(SEQ ID NO: 9)
CDR1-VL: KASQDISSYLS
(SEQ ID NO: 10)
CDR2-VL: RANRLLD,
and
(SEQ ID NO: 11)
CDR3-VL: LQYDEFPLT

or a functionally equivalent variant of said CDRs; and
b) an antibody (also called 4.4.2 herein) comprising
   the three heavy-chain CDRs of sequences:

(SEQ ID NO: 12)
CDR1-VH: GFNIEDTYMH
(SEQ ID NO: 13)
CDR2-VH: RIDPANGNTKYDPKFRD,
and
(SEQ ID NO: 14)
CDR3-VH: SYGNYDWYFDV

or a functionally equivalent variant of said CDRs, and
the three light-chain CDRs of sequences:

CDR1-VL:
(SEQ ID NO: 15)
SASSSVSYMH
CDR2-VL:
(SEQ ID NO: 16)
DTSKLAS,
and
CDR3-VL:
(SEQ ID NO: 17)
QQWSSHPLT

or a functionally equivalent variant of said CDRs.

In a more particular embodiment of the eighth aspect of the invention, the monoclonal antibody comprises
the three heavy-chain CDRs of sequences:

CDR1-VH:
(SEQ ID NO: 6)
GYTFTDYVLH,
CDR2-VH:
(SEQ ID NO: 7)
IDSSNIKYNENFRD,
and
CDR3-VH:
(SEQ ID NO: 8)
GDYDPFDY

or a functionally equivalent variant of said CDRs; and
the three light-chain CDRs of sequences:

CDR1-VL:
(SEQ ID NO: 9)
KASQDISSYLS
CDR2-VL:
(SEQ ID NO: 10)
RANRLLD,
and
CDR3-VL:
(SEQ ID NO: 11)
LQYDEFPLT

or a functionally equivalent variant of said CDRs.

As used herein, the term “treating” (or “treat” or “treatment”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders. The treatment of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). In the context of the present invention, the disorder or disease is cancer.

In the second medical use of the invention, the cancer is characterized by expressing of IL13Rα2, i.e. comprises cancer cells expressing the IL-13Rα2 protein with respect to a reference value. The term “interleukin-13 receptor subunit alpha-2” or “IL13Rα2”, as used herein, refers to a protein that binds IL-13 with high affinity. In humans it is encoded by the gene IL13Rα2a. The IL13Rα2 can be of any origin, for example human, bovine, murine, equine, canine, etc. In a particular embodiment, the IL13Rα2 is the human protein with the Uniprot accession number Q14627 (entry version 170, 27 Sep. 2017; sequence version 1, 1 Nov. 1996).

The term “cancer characterized by expressing IL13Rα2 ng”, as used herein, refers to the fact that the cancer comprises cells which which express IL13Rα2. The term “expressing IL13Rα2” or “express of IL13Rα2”, as used herein, refers to the level of the product of IL13Rα2 gene, that is, the levels of the messenger RNA or of the protein encoded by said gene. Nevertheless, in the context of the present invention, the detection of the IL13Rα2 protein expression is preferred The presence of the protein IL13Rα2 in a cancer sample or in a cell obtained from a cancer sample can be determined by methods well known in the art, for example, by means of a technique which comprises the use of antibodies with the capacity for binding specifically to IL13Rα2 (or to fragments thereof containing the antigenic determinants), or alternatively by means of a technique which does not comprise the use of antibodies such as, for example, by techniques based on mass spectroscopy. Illustrative examples of these techniques include, without limiting to, Western-blot or Western transfer, ELISA (enzyme-linked immunosorbent assay), RIA (radioimmuneassay), DAS-ELISA (double antibody sandwich ELISA), immunocytochemical and immunohistochemical techniques, techniques based on the use of protein biochips or microarrays that include specific antibodies or assays based on colloidal precipitation in formats such as dipsticks. Other ways of detecting and quantifying the IL13Rα2 include affinity chromatography techniques, ligand binding assays, mass spectrometry, etc. However, preferably, the detection of the protein expression is carried out by means of Western blot, ELISA, a protein array or a binding study. When an immunological method is used, any antibody or reagent that is known to bind to IL13Rα2 with high affinity can be used to detect the quantity thereof. On the market there are commercial antibodies against the IL13Rα2 that can be used in the context of the present invention with this purpose.

The second medical use of the invention relates to treatment of a cancer which is characterized by expressing of IL13Rα2. The term “cancer”, as used herein, refers to a disease characterized by uncontrolled cell division (or by an increase of survival or apoptosis resistance) and by the ability of said cells to invade other neighboring tissues (invasion) and spread to other areas of the body where the cells are not normally located (metastasis) through the lymphatic and blood vessels, circulate through the bloodstream, and then invade normal tissues elsewhere in the body. Depending on whether or not they can spread by invasion and metastasis, tumors are classified as being either benign or malignant: benign tumors are tumors that cannot spread by invasion or metastasis, i.e., they only grow locally; whereas malignant tumors are tumors that are capable of spreading by invasion and metastasis. Biological processes known to be related to cancer include angiogenesis, immune cell infiltration, cell migration and metastasis. As used herein, the term cancer includes, but is not limited to, the following types of cancer: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

In a particular embodiment of the second medical use the invention, the cancer is colon cancer, colorectal cancer, liver cancer, ovarian cancer, pancreatic cancer, glioblastoma or breast cancer.

In a particular embodiment the cancer is breast cancer, more particularly, triple-negative breast cancer. The term “triple-negative breast cancer” refers to a breast cancer characterized as estrogen receptor-negative, progesterone receptor-negative and human epidermal growth factor receptor-2-negative (FIER2-negative). The determination of negative status of the estrogen, progesterone, and Her2/neu expression is readily determined by one of skill in the art, e.g., in accordance with the current accepted guidelines

In a particular embodiment of the second medical use of the invention, the cancer is cancer metastasis, more in particular, colon cancer metastasis, colorectal cancer metastasis, liver cancer metastasis, ovarian cancer metastasis, pancreatic cancer metastasis, glioblastoma metastasis or lung cancer metastasis.

In a particular embodiment of the second medical use of the invention, the cancer metastasis is cancer metastasis from colorectal cancer or from breast cancer, particularly, triple-negative breast cancer. In a more particular embodiment, the cancer metastasis is liver cancer metastasis from colorectal cancer or lung cancer metastasis from breast cancer, particularly, from triple-negative breast cancer. In an even more particular embodiment, the cancer metastasis is liver cancer metastasis from colorectal cancer or lung cancer metastasis from breast cancer, particularly, triple-negative breast cancer, and the monoclonal antibody is the antibody comprising
the three heavy-chain CDRs of sequences:

CDR1-VH:
(SEQ ID NO: 6)
GYTFTDYVLH,
CDR2-VH:
(SEQ ID NO: 7)
IDSSNIKYNENFRD,
and
CDR3-VH:
(SEQ ID NO: 8)
GDYDPFDY

or a functionally equivalent variant of said CDRs; and
the three light-chain CDRs of sequences:

CDR1-VL:
(SEQ ID NO: 9)
KASQDISSYLS
CDR2-VL:
(SEQ ID NO: 10)
RANRLLD,
and
CDR3-VL:
(SEQ ID NO: 11)
LQYDEFPLT

or a functionally equivalent variant of said CDRs.

In the present invention, the terms “subject” and “individual” are used interchangeably. As used in the present document, the term “subject” or “individual” refers to all animals classified as mammals and includes, but is not limited to, farm and domestic animals, primates and humans, for example human beings, non-human primates, cows, horses, pigs, sheep, goats, dogs, cats or rodents. In a particular embodiment of the second medical use of the invention, the subject is a human being, male or female, of any age or race.

In a ninth aspect, the present invention relates to the monoclonal antibody or antigen-binding fragment, the immune cell comprising the chimeric antigen receptor, the antiserum, or the pharmaceutical composition of the invention, for use in the prevention of metastatic progression in a subject suffering from a cancer.

In a particular embodiment of the ninth aspect of the invention, the monoclonal antibody is selected from the group consisting of
a) an antibody (also called 5.5.4 herein) comprising
   the three heavy-chain CDRs of sequences:

CDR1-VH:
(SEQ ID NO: 6)
GYTFTDYVLH,
CDR2-VH:
(SEQ ID NO: 7)
IDSSNIKYNENFRD,
and
CDR3-VH:
(SEQ ID NO: 8)
GDYDPFDY

or a functionally equivalent variant of said CDRs; and
the three light-chain CDRs of sequences:

CDR1-VL:
(SEQ ID NO: 9)
KASQDISSYLS
CDR2-VL:
(SEQ ID NO: 10)
RANRLLD,
and
CDR3-VL:
(SEQ ID NO: 11)
LQYDEFPLT

or a functionally equivalent variant of said CDRs; and
b) an antibody (also called 4.4.2 herein) comprising
   the three heavy-chain CDRs of sequences:

CDR1-VH:
(SEQ ID NO: 12)
GFNIEDTYMH
CDR2-VH:
(SEQ ID NO: 13)
RIDPANGNTKYDPKFRD,
and
CDR3-VH:
(SEQ ID NO: 14)
SYGNYDWYFDV

or a functionally equivalent variant of said CDRs, and
the three light-chain CDRs of sequences:

CDR1-VL:
(SEQ ID NO: 15)
SASSSVSYMH
CDR2-VL:
(SEQ ID NO: 16)
DTSKLAS,
and
CDR3-VL:
(SEQ ID NO: 17)
QQWSSHPLT

or a functionally equivalent variant of said CDRs.

In a more particular embodiment of the ninth aspect of the invention, the monoclonal antibody comprises
the three heavy-chain CDRs of sequences:

CDR1-VH:
(SEQ ID NO: 6)
GYTFTDYVLH,
CDR2-VH:
(SEQ ID NO: 7)
IDSSNIKYNENFRD,
and
CDR3-VH:
(SEQ ID NO: 8)
GDYDPFDY

or a functionally equivalent variant of said CDRs; and
the three light-chain CDRs of sequences:

CDR1-VL:
(SEQ ID NO: 9)
KASQDISSYLS
CDR2-VL:
(SEQ ID NO: 10)
RANRLLD,
and
CDR3-VL:
(SEQ ID NO: 11)
LQYDEFPLT

or a functionally equivalent variant of said CDRs.

The term “prevention”, as used herein, refers to the capacity monoclonal antibody according, the antiserum, or the pharmaceutical composition of the invention, to avoid, minimize or hinder the metastatic progression of a cancer, in a particular embodiment, the metastatic progression of a cancer characterized by having increased expression of IL13Rα2 compared to a reference value.

The term “metastatic progression”, as used herein, refers to the process through which a tumor/cancer spreads to body tissues different than the primary site of tumor/cancer origin.

In a particular embodiment of the ninth aspect of the invention, the cancer is colon cancer, colorectal cancer, liver cancer, ovarian cancer, pancreatic cancer, glioblastoma or breast cancer, particularly, triple-negative breast cancer.

In a particular embodiment of the ninth aspect of the invention, the cancer metastasis is cancer metastasis from colorectal cancer or from breast cancer, particularly, triple-negative breast cancer. In a more particular embodiment, the cancer metastasis is liver cancer metastasis from colorectal cancer or lung cancer metastasis from breast cancer, particularly, from triple-negative breast cancer. In an even more particular embodiment, the cancer metastasis is liver cancer metastasis from colorectal cancer or lung cancer metastasis from breast cancer, particularly, from triple-negative breast cancer, and the monoclonal antibody is the antibody comprising
the three heavy-chain CDRs of sequences:

CDR1-VH:
(SEQ ID NO: 6)
GYTFTDYVLH,
CDR2-VH:
(SEQ ID NO: 7)
IDSSNIKYNENFRD,
and
CDR3-VH:
(SEQ ID NO: 8)
GDYDPFDY

or a functionally equivalent variant of said CDRs; and
the three light-chain CDRs of sequences:

CDR1-VL:
(SEQ ID NO: 9)
KASQDISSYLS
CDR2-VL:
(SEQ ID NO: 10)
RANRLLD,
and
CDR3-VL:
(SEQ ID NO: 11)
LQYDEFPLT

or a functionally equivalent variant of said CDRs.

In a tenth aspect, the invention relates to an in vitro method for designing a personalised therapy for a subject suffering from cancer, hereinafter “method of the invention”, comprising detecting the expression of IL13Rα2 in a cancer cell isolated from biological sample of the subject, wherein if the expression of IL13Rα2 is detected, then the therapy to be administered to the subject is the monoclonal antibody or antigen-binding fragment, the antiserum or the pharmaceutical composition of the invention.

The terms “subject”, “cancer” and “expression of IL13Rα2” have been defined above for previous inventive aspects and are applicable to the tenth aspect of the invention. Likewise, methods for determining the expression of IL13Rα2 has been explained in the above paragraphs.

The therapy is considered “personalised” when the compound (antibody of the invention) that is administered to the individual for treating a disease (cancer) is especially adapted to both the genotypic and phenotypic characteristics of the individual who is going to be treated, thereby avoiding the loss of time with non-effective therapies. In the present invention, the characteristic that determines the therapy that is going to be administered to the individual is the expression of the IL13Rα2 in a cancer cell.

As used herein, the term “biological sample” means any material comprising a nucleic acid. Examples of biological samples useful in the context of the present invention include, but without limiting to, blood and a biopsy sample of a cancer tissue (e.g. PBMCs (peripheral blood-derived mononuclear cells) such as neutrophils, monocytes). In a particular embodiment of the method of the invention, the biological sample is a biopsy sample.

In another particular embodiment of the method of the invention, alone or in combination with the previous particular embodiment, the subject is a human being. Said human being male or female, of any age or race.

In a particular embodiment, the cancer is colon cancer, colorectal cancer, liver cancer, ovarian cancer, pancreatic cancer or glioblastoma.

In a particular embodiment of the method of the invention of the invention, alone or in combination with the above particular embodiments, the cancer is cancer metastasis.

In a still more in particular, the cancer is colon cancer metastasis, colorectal cancer metastasis, liver cancer metastasis, ovarian cancer metastasis, pancreatic cancer metastasis or glioblastoma metastasis.

In a particular embodiment, the cancer metastasis is cancer metastasis from colorectal cancer. In a more particular embodiment, the cancer metastasis is liver cancer metastasis from colorectal cancer. In an even more particular embodiment, the cancer metastasis is liver cancer metastasis from colorectal cancer and the monoclonal antibody is the antibody comprising
the three heavy-chain CDRs of sequences:

CDR1-VH:
(SEQ ID NO: 6)
GYTFTDYVLH,
CDR2-VH:
(SEQ ID NO: 7)
IDSSNIKYNENFRD,
and
CDR3-VH:
(SEQ ID NO: 8)
GDYDPFDY

or a functionally equivalent variant of said CDRs; and
the three light-chain CDRs of sequences:

CDR1-VL:
(SEQ ID NO: 9)
KASQDISSYLS
CDR2-VL:
(SEQ ID NO: 10)
RANRLLD,
and
CDR3-VL:
(SEQ ID NO: 11)
LQYDEFPLT

or a functionally equivalent variant of said CDRs.

In an eleventh aspect, the present invention relates to the in vitro use of the monoclonal antibody, the antiserum, or the pharmaceutical composition of the invention, for drug screening.

Unless otherwise defined, all technical and scientific terms used herein have the same meaning as commonly understood by one of ordinary skilled in the art to which this invention belongs. Methods and materials similar or equivalent to those described herein can be used in the practice of the present invention. Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples, drawings and sequence listing are provided by way of illustration and are not intended to be limiting of the present invention.

### BRIEF DESCRIPTION OF THE DRAWINGS

FIG. 1. Functional screening and selection of IL13Rα2 D1-specific antibodies to inhibit IL13-promoted cell invasion. For antibody screening, KM12SM cells were subjected to inhibition of cell invasion through Matrigel in the presence of A) the polyclonal serum of each animal and B) supernantants after hybridoma fusion. C) Same supernatants were tested by indirect ELISA against the uncoupled IL13Rα2 peptide. D, E) Preselected clones #4 and #5 and further subclones were tested in cell invasion inhibition assays either in KM12SM or SW620 cells. F) Cell invasion assays with a chicken antibody (GC-13) prepared against a similar D1 peptide.

FIG. 2. Functional characterization of anti-IL13Rα2 D1 peptide antibodies. A) Sandwich ELISA of anti-IL13Rα2 mAbs 4.4.2 and 5.5.4 vs biotin-labelled peptide. B) MAbs 4.4.2 or 5.5.4 significantly inhibited the binding of IL-13 to immobilized IL13Rα2 using a competition ELISA (*p<0.05; **p<0.01; ***p<0.001). C) IL13Rα2 immunoprecipitation using IL13Rα2 D1 mAbs in KM12SM cells (2K8, commercial IL13Rα2 Ab, Ctrl: Negative control). D) Flow cytometry using IL13Rα2 D1 mAbs. E) Immunofluorescence analysis of IL13Rα2 expression and staining using IL13Rα2 D1-specific mAbs in KM12SM, KM12C, SW620 and SW480 colorectal cancer cells.

FIG. 3. Inhibition of IL-13 pro-metastatic capacities. Metastatic KM12SM and SW620 colorectal cancer cells were treated with IL-13 (10 ng/ml) and subjected to cell adhesion, wound healing, invasion and MTT assays in the presence of the anti-IL13Rα2 D1-specific mAbs and chicken antibody GC-13. Anti-IL13Rα2 D1 peptideantibodies significantly inhibited cell invasion, adhesion, migration and proliferation triggered by IL-13 (0p<0.05; 00p<0.01; 00p<0.001).

FIG. 4. Molecular pathway analysis of cell signaling inhibition by IL13Rα2 D1-specific antibodies. KM12SM, SW620, RKO and CT-26 colorectal cancer cell lines were treated with IL-13 for the indicated times in serum-free DMEM in absence or presence of IL13Rα2 D1-specific mAbs. Cell extracts were collected at the indicated times and analyzed by western blot with antibodies against FAK, SRC, AKT, ERK1/2 and their phosphorylated forms. RhoGDI was used as a loading control. IL13Rα2 D1-specific antibodies inhibited phosphorylation of IL-13/IL13Rα2 signaling mediators in a time and cell type-dependent mode.

FIG. 5. Effect of IL13Rα2 D1 mAbs on IL-13 signaling pathways, receptor internalization and degradation. A) HT29 cells expressing IL13Rα1 and IL13Rα2 receptors were treated or not with IL-13 in presence of the IL13Rα2 D1-specific mAbs and then analyzed by western blot to detect phospho-SRC and phospho-STAT6. Total SRC and STAT6 were used as loading controls. Both mAbs caused a clear inhibition of SRC activation but not IL13Rα1-mediated STAT6. B) Inhibition of IL-13-promoted receptor internalization by D1-specific mAbs. All D1-specific antibodies inhibited IL-13-induced receptor internalization at a significant extent (^{⋄⋄}p<0.01; ^{⋄⋄⋄}p<0.001) and the internalization of the receptor by the mAbs compared with a control antibody (***p<0.001). C) Inhibition of receptor degradation after IL13-promoted internalization by D1-specific mAbs in KM12SM and SW620 cells. IL13Rα2 degradation was detected by western blot and quantified by densitometric analysis (*p<0.05; **p<0.01; ***p<0.001).

FIG. 6. MAb 5.5.4 protects mice from liver metastasis induced by IL13Rα2-positive cancer cells. A) Swiss nude mice were inoculated intrasplenically with metastatic KM12SM cells and 48 h later mice were treated with 4.4.2 and 5.5.4 mAbs (50 mg/Kg of weight, divided in 7 doses) administered intravenously. Kaplan-Meier survival results indicated a significantly enhanced survival after treatment with the 5.5.4 mAb (***p<0.001). Representative pictures of livers from the inoculated mice after necropsy are shown. B) Swiss nude mice were treated as in A, but only mAb 5.5.4 was used for the treatment. Kaplan-Meier analysis indicated a complete survival of treated mice (***p<0.001). C) Swiss nude mice were inoculated intra-tail vein with mouse CT-26 cells and 48 h later treated with 7 doses of mAb 5.5.4 or 4 doses of irinotecan. According to Kaplan-Meier analysis mice did not show a significant survival in any of the treated groups (p=0.126). Finally, D) Swiss nude mice were inoculated intrasplenically with metastatic KM12SM cells and 7 days later we started the treatment with 5.5.4 at the same dose. Treated mice showed a complete survival (p=0.018).

FIG. 7. IL13Rα2 expression in triple negative breast cancer cells and survival of mice treated with Mab 5.5.4. Expression of IL13Rα2 A) in the total extract of KM12SM 231 and LM2 cells by WB and B) in membrane determined by flow cytometry. C) Kaplan-Meier survival curves for NSG inoculated with LM2 cells intravenously. Starting 48 hours after inoculation, Mab 5.5.4 was administered intravenously for 2 weeks. When signs of disease were detected, mice were sacrificed and examined for gross lung metastases. Survival was significantly improved with AM 5.5.4 (*p<0.05) D) Representative images of livers and lungs of inoculated mice after necropsy **p<0.01).

### EXAMPLES

### I—Materials and Methods

### Cell Lines, Reagents and Antibodies

Highly metastatic KM12SM human colon cancer cells were obtained from Dr. I. Fidler (MD Anderson Cancer Center. Houston, TX, USA). Human SW480 and RKO colon cancer cell lines were purchased from the ATCC. Human SW620 colon cancer cell line was purchased from the ECACC. Human HT29 colon cancer cells were obtained from Dr. Mollinedo (CIB-CSIC). All cell lines were passaged fewer than 6 months after purchase for all the experiments. KM12SM were authenticated in our laboratory every 6 months. Murine CT26 colon carcinoma cell lines wereobtained from Prof. Caroline Saucier (Université de Sherbrooke (Québec), Canada). All cell lines were cultured in DMEM (Invitrogen) containing 10% FCS (Invitrogen) and antibiotics at 37° C. in a 5% CO₂ humidified atmosphere.

Human IL-13 was used at 10 ng/ml and was purchased from PeproTech (UK). Irinotecan (Selleckchem. Germany) was used at 50 mg/Kg of weight. Antibodies used in the experiments are listed in table 1.

TABLE 1

| Antibody | Target | Application | Supplier |
|---|---|---|---|
| ab55275 | IL13Rα2 | Flow cytometry | Abcam |
| PA5-46976 | IL13Rα2 | Flow cytometry | Thermo Fisher |
| clone 47 | IL13Rα2 | Blocking | Described in Balyasnikova |
| | | | et al. 2012 J. Biol. Chem. |
| | | | 287: 30215-30227. |
| 2K8 | IL13Rα2 | Immunoprecipitation, | Santa Cruz Biotechnologies |
| | | western blotting | |
| A-20 | RhoGDIα | Western blotting | Santa Cruz Biotechnologies |
| D7A5 | Phospho-Tyr1068 | Western blotting | Cell Signaling Technology |
| | EGFR | | |
| #4695 | p44/42 MAP Kinase | Western blotting | Cell Signaling Technology |
| #9106 | pThr202-p44/ | Western blotting | Cell Signaling Technology |
| | pTyr204-p42 MAPK | | |
| A-17 | FAK | Western blotting | Santa Cruz Biotechnologies |
| FAK | pTyr397-FAK | Western blotting | BD Transduction Laboratories |
| (pY397) 14 | | | |
| AF3389 | SRC | Western blotting | R&D Systems |
| #6943 | pTyr416 Src | Western blotting | Cell Signaling Technology |
| | family | | |
| #2105 | pTyr530 Src | Western blotting | Cell Signaling Technology |
| #2920 | AKT | Western blotting | Cell Signaling Technology |
| #3787 | pSer473-AKT | Western blotting | Cell Signaling Technology |
| #9364 | pSTAT6 | Western blotting | Cell Signaling Technology |
| #9362 | STAT6 | Western blotting | Cell Signaling Technology |

### Peptide Design, Immunization, Preparation and Selection of Anti-IL13Rα2 Mouse Monoclonal Antibodies

For mouse immunization, a 19-mer IL13Rα2 peptide (IGSETWKTIITKNLHYKD-Cys (SEQ ID NO: 3)) comprising the sequence GSETWKTIITKN (SEQ ID NO: 2) was synthesized using F-moc Solid Phase Peptide Synthesis (Proteogenix, France) and conjugated to ovalbumin (OVA). Animal experimental protocols were approved by the ethics committee of the Instituto de Salud Carlos III (CBA22_2014-v2) and Community of Madrid (PROEX 278/14). Three female Balb/c mice were immunized 3 times each intraperitoneally (i.p.) using OVA-conjugated IL13Rα2 peptide—the first time with 50 μg of peptide-OVA emulsified in Freund's complete adjuvant (FCA) and then twice with 25 μg of peptide-OVA emulsified in Freund's incomplete adjuvant (FIA), with intervals of 15 days between each dose. The mouse myeloma SP2/O-Ag-14 cell line was selected for the fusion and mAbs were generated according to standard procedures. Clone selection was carried out by indirect ELISA against the unconjugated IL13Rα2 peptide according to standard procedures and functional screening of preselected clones in invasion assays. Selected mAbs were purified by affinity chromatography using Protein G columns, and dialyzed against PBS for final testing and characterization. Isotype of final clones was determined with Rapid ELISA Mouse mAb Isotyping Kit (Pierce).

### Chicken Antibodies

Alternatively, a 13-mer IL13Rα2 synthetic peptide (GSETWKTIITKNC (SEQ ID NO: 4)) (Proteogenix, France) was further conjugated to OVA. All animal experiments in this study were conducted according to the European Union Directive 2010/63/EU. Two (2) White Leghorn chickens were immunized and boosted under the wing, in the armpit, during 9 weeks with the OVA-conjugated IL13Rα2 peptide according to standard procedures. Purification of chicken polyclonal antibodies from collected eggs was performed by precipitation with sodium sulfate followed by affinity chromatography against the IL13Rα2 13-mer peptide of the IgY fraction, to finally obtain the specific chicken anti-IL13Rα2 antibodies. Purified antibodies were dialyzed against PBS for final testing and usage.

### Capture ELISA

Maxisorp 96-well microtiter plates (Nunc) were coated by addition of 100 μL/well of a 5 μg/mL solution of anti-IL13Rα2 mAbs 4.4.2 or 5.5.4 in carbonate buffer (50 mmol/L, pH 9.6) and overnight incubation at 4° C. Coated plates were washed three times with washing buffer (PBS with 0.05% (v/v) Tween-20) and then 150 μL of 2% BSA in PBS was added to each well to reduce non-specific binding. The plate was washed three times with washing buffer and 100 μL of Biotin-GGGSETWKTIITKN (SEQ ID NO: 5) peptide was added to coated wells in log 2 dilution from a 2 μg/mL standard. An irrelevant biotin labelled-protein was used as control. Plates were incubated at 37° C. for 1 hour and washed three times with washing buffer. Next, 100 μL of a 1/2000 dilution of Streptavidin-HRP conjugated (Southern Biotechnology) was added to each well and plates were incubated for 30 minutes at RT. Finally, plates were washed 5 times with washing buffer and 100 μL/well of TMB substrate solution (Sigma Aldrich) was added. Colour development was stopped after 10 minutes incubation by addition of 50 μL/well of H₂SO₄ (2N). The absorbance was measured at 450 nm.

### Competition ELISA

Microtiter plates (Maxisorp, Nunc) were coated overnight with 1 μg/mL of purified IL13Rα2 ectodomain (Protein Alternatives SL. Spain). After washing three times with PBS, plates were blocked with 3% skimmed milk in PBS for 2 hours at room temperature. Then, IL-13 (0.1 μg/mL) and mAbs at different concentrations (1-50 μg/well) were added to the plates and incubated for 1.5 hours at room temperature. An irrelevant antibody was used as a control. After washing, peroxidase-labeled anti-IL-13 antibody (Abexxa, Cambridge, UK) (0.4 μg/mL) was added for 1.5 hours at room temperature. Color was developed with 3, 3′, 5, 5′-tetramethylbenzidine substrate (Sigma-Aldrich). The reaction was stopped with 1 M HCl, and absorption measured at 450 nm.

### Antibody Confocal Microscopy

Colorectal KM12SM, KM12C, SW620 and SW480 cells were cultured on Matrigel-coated cover slides, fixed with 4% paraformaldehyde in PBS. After washing, cells were incubated overnight with primary and control antibody at 30 μg/mL in PBS with human gamma-globulin (40 μg/mL) at 4ºC. Cells were then incubated 40 minutes with secondary antibodies coupled with Alexa-488 and 4,6-diamidino-2-phenylindole (DAPI). Samples were mounted with Mounting Fluorescence Medium (Dako, Copenhagen, Denmark) and images were captured using a TCS-SP5-AOBS confocal microscope with 63× oil immersion objective.

### Flow Cytometry

Cells were detached with 2 mM EDTA in PBS, incubated at 4° C. with primary antibodies (10 μg/mL) for 30 minutes, washed and incubated with Alexa-488-labelled-secondary antibodies (Agilent). Fluorescence was analyzed in a Coulter Epics XL cytofluorimeter (Beckman-Coulter). Mean fluorescence intensity of 10,000 analyzed cells is shown for each cell type.

### Western Blot Analysis and Immunoprecipitation

Cells were detached, washed, and lysed with protease and phosphatase inhibitors in lysis buffer (1% Igepal, 50 mmol/L NaCl, 2 mmol/L MgCl₂, 10% glycerol, complete mini protease inhibitors (Roche) and phosphatase inhibitors cocktails 2 and 3 (Sigma-Aldrich) in water. Protein extracts were separated in SDS-PAGE, transferred to nitrocellulose membranes, incubated with primary antibodies (table 1), followed by incubation with either HRP-anti-mouse IgG (Thermo Scientific) or HRP-anti-rabbit IgG or HRP-anti-goat IgG (Sigma-Aldrich). Reactive proteins were visualized with SuperSignal West Pico Chemiluminescent Substrate (Thermo Scientific).

For immunoprecipitation, 500 μg of cell lysates were incubated with 3 μg of the indicated antibodies. The immunocomplex was captured by adding 25 μL of Protein G-sepharose beads (Sigma-Aldrich). After washing, samples were resuspended in Laemmli buffer, boiled for 5 minutes, centrifuged and subsequently loaded onto SDS-PAGE for western blot analysis. As a control, we incubated the lysates with an unrelated IgG coupled to Sepharose beads to discard unspecific protein binding.

### IL13Rα2 Internalization

Cells were starved for 3 hours, detached with 2 mM EDTA, incubated 45 minutes with IL-13 in the presence or absence of the antibodies (15 μg/ml) at 37 ºC. Then, cells were washed in ice cold PBS. Primary antibodies were added for 1 hour, followed by the secondary antibodies for 30 minutes at 4ºC. Cells were analyzed in the cytofluorimeter as previously described (Bartolome R A et al. Br J Cancer 2018; 119: 940-9).

### Cell Signaling

Cells were incubated 3 hours in serum-free DMEM, detached with 2 mM EDTA, washed and treated with anti-IL13Rα2 D1 mAbs (15 μg/ml) for 15 minutes and then treated with IL-13 (10 ng/mL) at different times (0, 10, 60 minutes). Ice cold PBS was used to stop the reaction and, finally, cells were lysed and subjected to western blot as before

### Cell Adhesion

Cancer cells were kept in serum-free medium for 3 hours, detached with 2 mM EDTA in PBS and labelled with BCECF-AM (Molecular Probes, USA). Following a 10-minutes incubation in serum-free medium with anti-IL13Rα2 peptide mAbs (15 μg/mL) and/or IL-13 (10 ng/mL), 6×10⁴ cells were loaded into 96-well plates previously coated with Matrigel (0.4 μl/well) (BD Biosciences) and blocked with 0.5% BSA. After 25-minutes incubation of the loaded plates at 37° C., non-adhesive cells were removed by three washes with serum-free medium. Adhesive cells were lysed with 1% SDS in PBS and cell adhesion was quantified in a POLARstar Galaxy fluorescence analyser (BMG Labtech, Ortenberg, Germany).

### Wound Healing

1 mm-wide scratch was made across a confluent monolayer of cancer cells in 24-well plates previously coated with Matrigel (0.2 μg/well). Plates were incubated in 0.5% serum medium with anti-IL13Rα2 peptide mAbs (15 μg/mL) and/or IL-13 (10 ng/ml) for 24 hours at 37ºC. Distance covered by cells was estimated using pictures of the scratches taken at times 0 and 24 hours. Migration speed was obtained as covered distanced divided by 24 hours and divided by 2 (as cell migrated by two sides of the wound).

### Cell Invasion Assays

For invasion assays, 6.5 mm diameter Transwell filters with 8 μm pores (Corning Inc., ME, USA) were filled with a mix of 22 μL of serum-free DMEM and 11 μL of Matrigel (BD Biosciences). When Matrigel solidified, 6×10⁴ cells in 200 μL serum-free DMEM were loaded in the upper compartments of Traswells in the presence of anti-IL13Rα2 peptide or control mAbs (15 μg/mL); whereas the lower compartments were filled with IL-13 (10 ng/ml) in 700 μL of serum-free DMEM. After 48 hours, cells which did not reach the lower surface of the filters were removed, while migrated cells were fixed with 4% paraformaldehyde, dyed with crystal violet, and observed under a microscope. The number of invasive cells to IL-13 in presence of control antibodies was considered as 100% of IL-13-triggered cell invasion.

### Cell Proliferation

Cell proliferation assays were carried out as previously described (Bartolome R A et al. Br J Cancer 2018; 119: 940-9).

### Experimental Metastasis in Nude Mice

The Ethics Committees of the CSIC and Community of Madrid approved all protocols used in animal experimentation (PROEX 252/15). Swiss nude mice (Charles River) (n=6 per condition) were inoculated in the spleen with 1.5×10⁶ KM12SM cells in 0.1 mL PBS. The day after inoculation, spleens were removed to avoid local growth of the tumor cells. Then, these mice were treated intravenously with anti-IL13Rα2 mAb 5.5.4 or chicken antibody GC-13 or control antibodies. Antibodies were used at 50 mg/Kg of weight, divided in 7 doses during 2 weeks, starting 2 days after inoculation, or after 7 days for the established metastasis model. Mice were daily inspected for signs of disease, such as abdominal distension, locomotive deficit, or tumor detectable by palpation. When signs were visible, mice were euthanized, subjected to necropsy, and inspected for metastasis in liver. For lung metastasis, mice were inoculated in the tail vein with 1×10⁵ mouse CT-26 cells in 0.1 mL PBS and treated intravenously with the 5.5.4 mAb as indicated before or with Irinotecan (50 mg/Kg of weight divided in 4 doses during 2 weeks). When signs of disease were visible, mice were euthanized, subjected to necropsy, and inspected for metastasis in liver or lungs.

### II—Results

Antibody Selection with the Capacity for Blocking IL13Rα2-Mediated Invasion

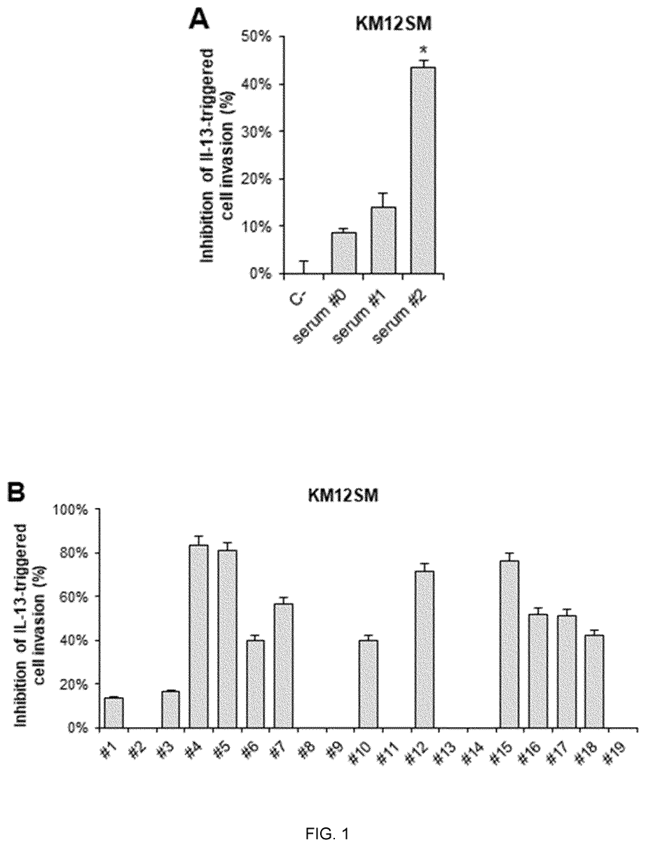

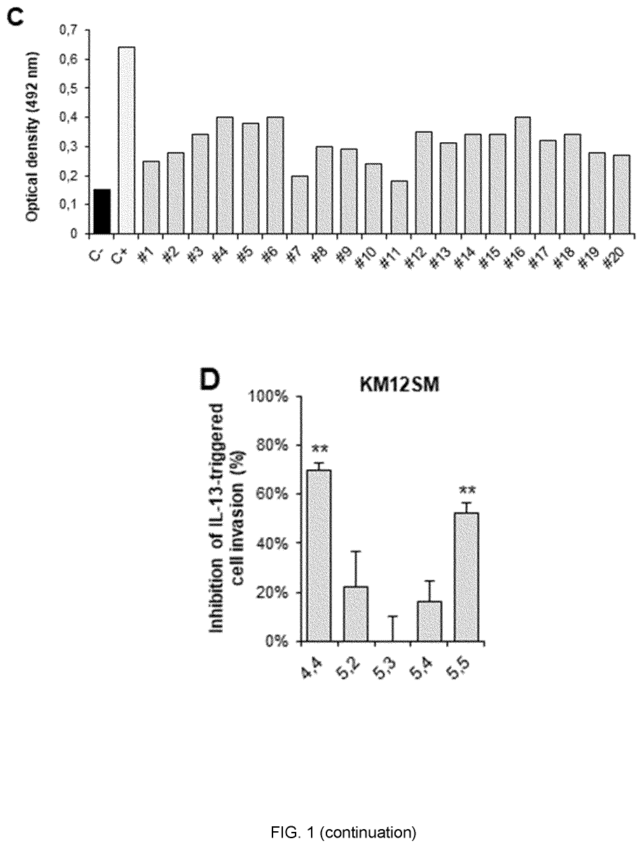

Mice were immunized with a 19-mer IL13Rα2-peptide (SEQ ID NO: 3) coupled to OVA. For hybridoma selection, a functional screening was implemented to test the antibody capacity for blocking the IL-13/IL13Rα2-promoted cell invasion through Matrigel. As a first step, pre-hybridoma fusion mouse sera were tested. Serum #2 showed a 40% inhibition of the cellular invasive capacity (FIG. 1A). This mouse was selected for spleen fusion and antibody production after a final boost with a recombinant IL13Rα2-Fc protein. Then, 20 antibody clones were screened by their capacity to inhibit invasion (FIG. 1B) and peptide recognition using ELISA (FIG. 1C). Two hybridoma supernatants (#4-5) inhibited >80% invasion of KM12SM cells and were selected for further subcloning. ELISA testing against the uncoupled peptide offered significant binding values for both supernatants. After further subcloning, clones 4.4 and 5.5 inhibited invasion by more than 50% and were selected for final clone selection (FIG. 1D). Final testing of eight clone supernatants resulted in the selection of two clones, 4.4.2 and 5.5.4, that inhibited IL-13-mediated cell invasion more than 60% in KM12SM and SW620 (FIG. 1E). These two mAbs were isotyped (4.4.2: IgG2b/K, 5.5.4: IgG1/K) and used for further experiments. Chicken antibody (GC-13) prepared against the same peptide also exhibited strong invasion inhibition (FIG. 1F). The mRNA was isolated from both hybridomas and the cDNAs were sequenced according to standard procedures. We have identified the following sequences: The antibody 5.5.4 comprises
the three heavy-chain CDRs of sequences SEQ ID NO: 5 (CDR1-VH: GYTFTDYVLH), SEQ ID NO: 6 (CDR2-VH: IDSSNIKYNENFRD) and SEQ ID NO: 7 (CDR3-VH: GDYDPFDY) and
the three light-chain CDRs of sequences SEQ ID NO: 8 (CDR1-VL: KASQDISSYLS), SEQ ID NO: 9 (CDR2-VL: RANRLLD) and SEQ ID NO: 10 (CDR3-VL: LQYDEFPLT).

The antibody 4.4.2 comprises
the three heavy-chain CDRs of sequences SEQ ID NO: 11 (CDR1-VH: GFNIEDTYMH), SEQ ID NO: 12 (CDR2-VH: RIDPANGNTKYDPKFRD) and SEQ ID NO: 13 (CDR3-VH: SYGNYDWYFDV), and
the three light-chain CDRs of sequences SEQ ID NO: 14 (CDR1-VL: SASSSVSYMH), SEQ ID NO: 15 (CDR2-VL: DTSKLAS) and SEQ ID NO: 16 (CDR3-VL: QQWSSHPLT).
   Characterization of IL13Rα2 D1-Specific mAbs

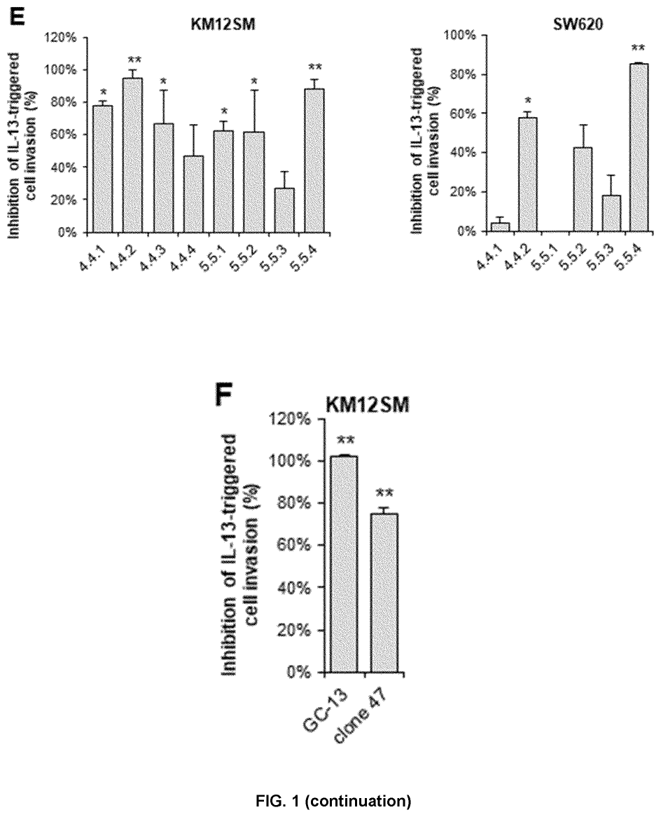

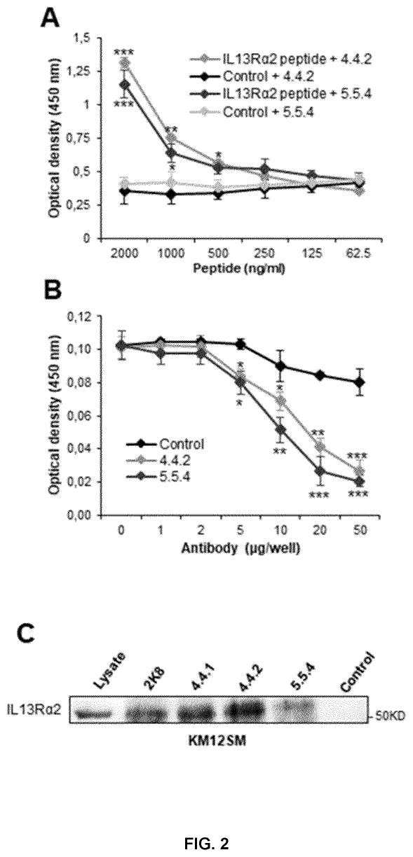

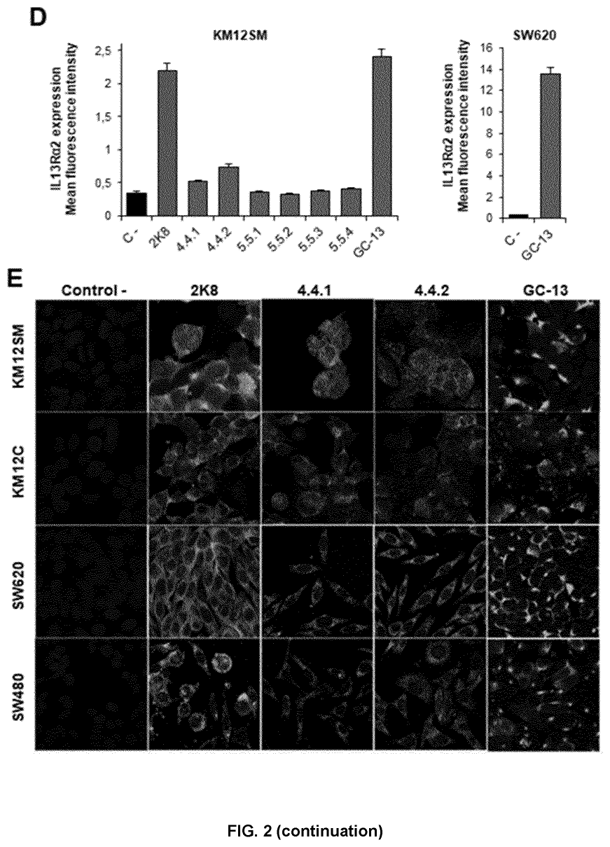

MAbs 4.4.2 and 5.5.4 specificity was confirmed by using a capture ELISA between the free peptide and the biotinylated peptide for the binding to the mAbs coated at 5 μg/mL. A significant competition up to 500 ng/ml was observed for the biotinylated peptide respect to the control for both mAbs (FIG. 2A). Furthermore, we performed a competition assay between the two mAbs and the recombinant IL-13 for the binding to the IL13Rα2 receptor. MAbs blocked IL-13 binding to IL13Rα2 starting at 5 μg/well, with a complete inhibition at 50 μg/well (FIG. 2B). A control antibody only exhibited a minor competition (<20%) at high concentrations. Both mAbs were negative by western blot (data not shown) but positive for IL13Rα2 immunoprecipitation (FIG. 2C). Flow cytometry results were negative except for a weak reactivity of the 4.4.2 mAb with KM12SM cells when compared to the chicken antibody and a commercial antibody (FIG. 2D). Finally, we tested the reactivity of mAbs 4.4.1, 4.4.2, 5.5.4 and chicken antibodies with different CRC cell lines using confocal microscopy. Commercial antibody 2K8 was used as positive control. Whereas mAbs 4.4.1 and 4.4.2 and commercial antibody exhibited membrane and cytoplasmic staining in all cell lines (FIG. 2E), 5.5.4 was negative (data not shown). In contrast, chicken antibody was more membrane specific. In summary, IL13Rα2 D1 peptide-specific mAbs exhibited a relatively weak reactivity with IL13Rα2 using conventional techniques. However, they appear to recognize an IL13Rα2 conformation required for cell invasion, which would confer a high functional selectivity to their use. Therefore, mAbs 4.4.2 and 5.5.4 were selected for further testing of their neutralizing capacity.

IL13Rα2 D1-Specific mAbs Inhibit Metastatic Properties in Colorectal Cancer Cells

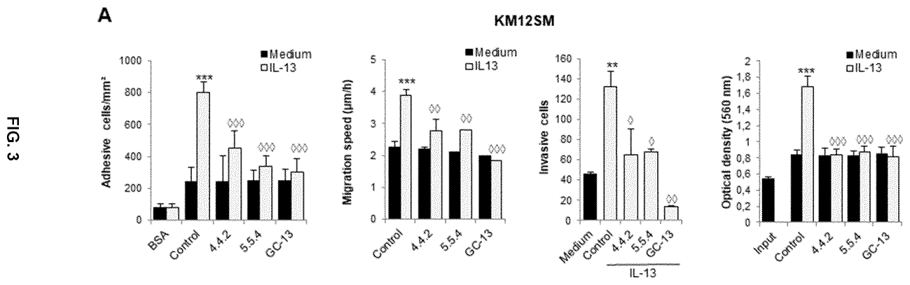

Next, we examined the capacity of mAbs 4.4.2 and 5.5.4 to inhibit IL13-mediated metastatic capacity in KM12SM and SW620 cells, which differ in the levels of IL13Rα2 expression (KM12SM>SW620) and phenotypic properties (epithelial vs mesenchymal). Overall, there was a significant similarity between the results obtained in both cell lines (FIG. 3). Both mAbs inhibited at a similar extent cell adhesion, migration, invasion and proliferation in the metastatic colorectal cancer cell lines. Interestingly, GC-13 chicken antibody prepared against a 13-mer IL13Rα2 synthetic peptide SEQ ID NO: 4 also showed excellent blocking properties for IL-13 mediated metastatic capacity (FIG. 3). Together, these results support the capacity of D1-specific mAbs for blocking IL-13-triggered cell invasion, migration and adhesion in colorectal cancer cell lines positive for IL13Rα2 expression.

IL13Rα2 D1-Specific mAbs Inhibit Ligand-Induced Phosphorylation of Downstream Signaling Molecules.

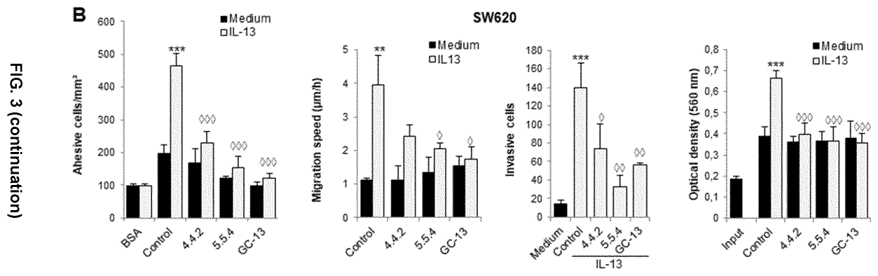

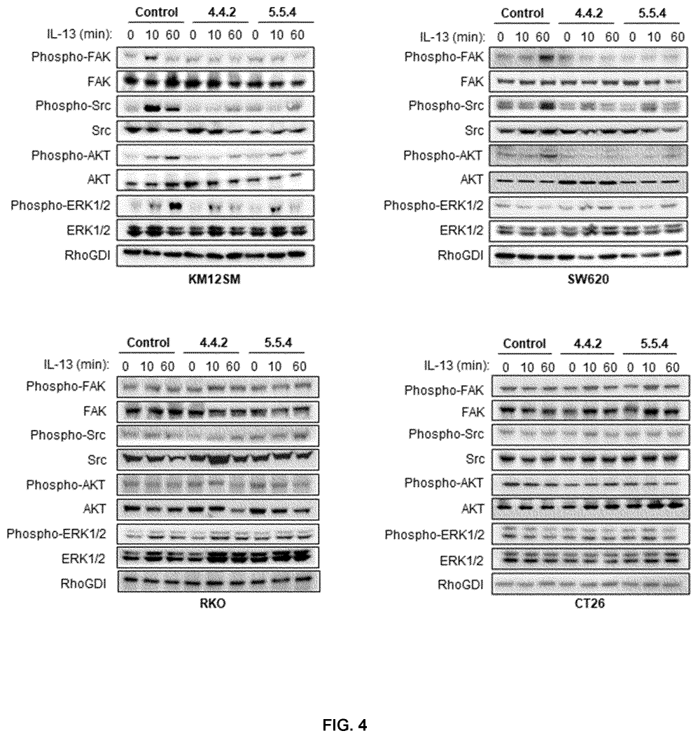

To further explore the alterations in the IL-13/IL13Rα2 signaling pathway, human KM12SM, SW620, RKO and murine CT-26 cell lines were treated with the two mAbs or a control IgG. These cell lines were chosen because they exhibited different levels of IL13Rα2 expression. RKO and mouse CT-26 cell lines are aggressive, poorly differentiated cell lines characterized by the lack of IL13Rα2 membrane expression. In KM12SM cells, a swift activation of pFAK and pSrc (10 minutes) was observed, followed by slower pAKT and pERK activation (60 minutes). Both mAbs diminished the activation of FAK, Src, AKT and ERK kinases (FIG. 4), which correlate with the observed decrease in cell adhesion, invasion and proliferation. In SW620 cells, FAK, Src and AKT inhibition occurred after a late activation at 60 minutes. No activation of pERK was observed in SW620 and RKO cells, which might be explained by the presence of KRAS and BRAF mutations in SW620 in RKO, respectively (Berg KCG et al. Mol Cancer 2017; 16:116). As expected, due to the lack of IL13Rα2 expression, RKO and murine CT-26 cells did not respond to IL-13 in the 60 minutes period. Therefore, no effect was observed for the mAbs on IL-13-mediated signaling in RKO and CT-26 cells. In summary, mAbs inhibited FAK, Src and AKT signaling after blocking the IL-13/IL13Rα2 axis in the metastatic cell lines expressing IL13Rα2. Downstream signaling inhibition correlates with the initial levels of IL13Rα2 membrane expression and the presence of mutations in the RAS-RAF-ERK cascade.

IL13Rα2 D1 mAbs Inhibit IL13Rα2 Activation but not IL13Rα1 Signaling

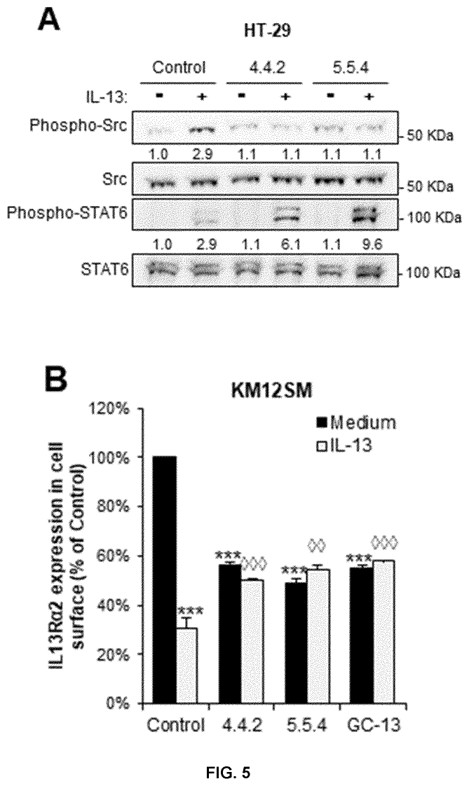

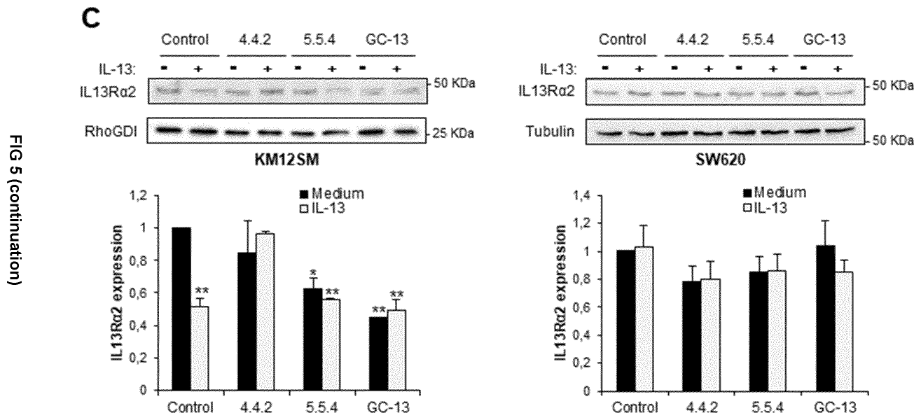

IL-13 signaling pathway involves the use of two different receptors. The canonical IL-13 receptor involves a heterodimer of IL13Rα1 with IL4Ra and signaling occurs through STAT6 activation, whereas IL13Rα2-mediated signaling is SRC-mediated, STAT6-independent. To examine the selectivity of the antibody interaction, we investigated the specificity of 4.4.2 and 5.5.4 to block both IL-13 receptors through the inhibition of the SRC and STAT6 activation pathways in the HT-29 cell line, which expresses both receptors in contrast to KM12SM or SW620 cell lines (Bartolome R A et al. Br J Cancer 2018; 119:940-9). Both antibodies inhibited SRC, but not STAT6 activation (FIG. 5A). Therefore, mAbs 4.4.2 and 5.5.4 selectively block the IL-13 binding to IL13Rα2 but not to IL13Rα1. This specificity might be highly relevant for those diseases that involve IL13Rα2 signaling but not IL13Rα1.

IL13Rα2 D1 mAbs Prevent the Internalization Capacity of IL-13 Binding to IL13Rα2

Receptor internalization is involved in signaling regulation. To test this notion, we explored the effect of mAbs on the IL13Rα2 internalization and degradation promoted by IL-13 using flow cytometry and western blot. IL-13 triggered a significant internalization of IL13Rα2 that was partially inhibited after treatment with D1-specific antibodies (FIG. 5B). However, both mAbs induced certain degree of receptor internalization. Regarding receptor degradation, we found some differences after antibody treatment. So, 5.5.4-treated cells showed more receptor degradation than 4.4.2-treated cells in KM12SM cells (FIG. 5C). This effect was more visible in KM12SM cells than in SW620 probably due to the different levels of expression. Therefore, mAb 5.5.4 seems to play a dual effect on IL13Rα2. On the one hand, 5.5.4 exhibits an antagonist activity on IL-13 binding, but on the other appears to cause an agonist-like effect after antibody binding, which induces a direct degradation of the receptor without activation.

### Monoclonal Antibody 5.5.4 is a Potent Inhibitor of Colorectal Cancer Metastasis

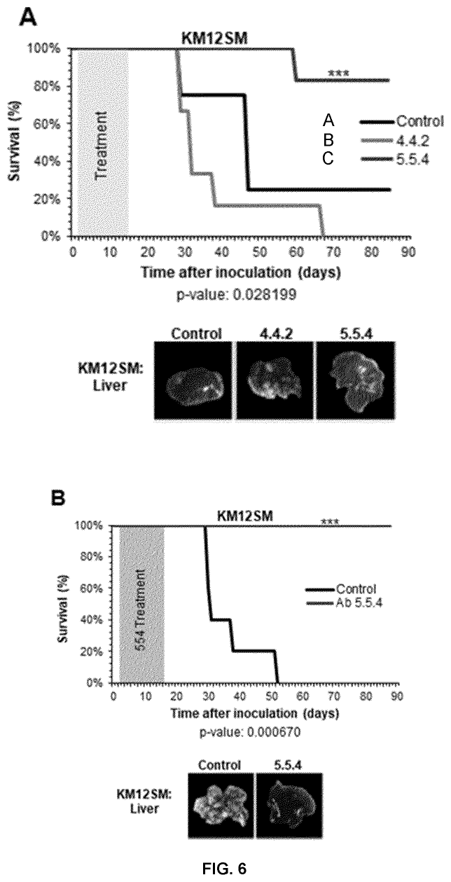

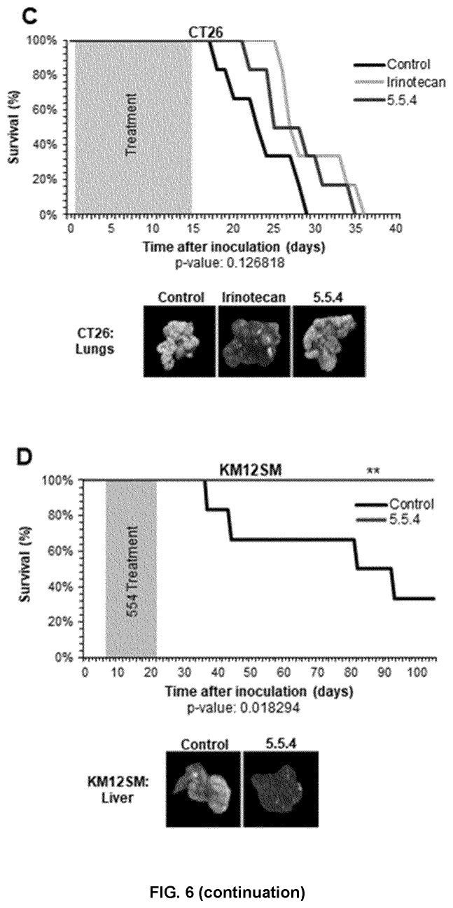

Finally, we evaluated the capacity of mAbs 4.4.2 and 5.5.4 to protect mouse against colorectal cancer metastasis. Swiss nude mice were inoculated into the spleen with KM12SM cells to induce liver colonization through the hepatic portal vein. Then, spleens were removed and mice (n=6) were treated with each mAb individually for 2 weeks, with a total dose of 50 mg of antibody per kg of mouse weight. Kaplan-Meier survival results indicated a quite different response to the antibody treatment, despite both mAbs were equally effective on blocking invasion capacity. Whereas mAb 4.4.2 slightly improved mice survival respect to the control group, mAb 5.5.4 conferred a strong protection, with 80% of the mice surviving the end-point without apparent metastatic lesions (FIG. 6A). To further confirm the neutralizing activity of mAb 5.5.4, we performed a second in vivo experiment. All mice treated with the mAb 5.5.4 survived to the inoculation of metastatic KM12SM cells without development of liver metastatic nodules (FIG. 6B). To demonstrate that this protection was related to the IL13Rα2 expression levels, we tested intravenous inoculation of CT-26 for lung metastasis treatment. Since these cells do not express IL13Rα2, after using either mAb 5.5.4 or irinotecan as treatments, mice survival to lung metastasis was not significantly improved (FIG. 6C). To note that irinotecan, a drug widely used in CRC chemotherapy, also failed to provide significant protection against CT-26 cells. Finally, we tested the effectiveness of 5.5.4 in established liver metastasis. In this experiment, antibody treatment started 7 days after the intra-splenic inoculation of the cells. All mice receiving the mAb 5.5.4 survived without metastasis development (FIG. 6D). In summary, mAb anti-IL13Rα2 5.5.4 has shown the capacity to protect mice against IL13Rα2 positive differentiated liver metastasis, including established metastasis. These results suggest a potential therapeutic application of mAb 5.5.4 in colorectal cancer.

### Therapeutic Effect of MAb 5.5.4 in Triple Negative Breast Cancer Metastasis

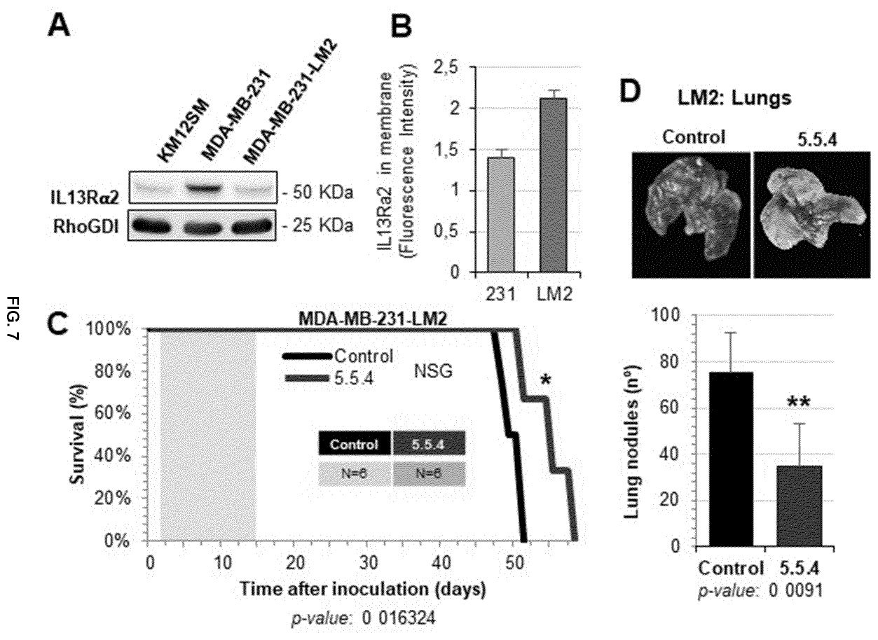

To verify a possible application of anti-IL13Rα2 mAbs in other types of tumors, mAb 5.5.4 was tested in the triple-negative breast cancer line MDA-MB-231-LM2. First, we studied the expression of IL13Rα2 by WB. It was observed that the parental line MDA-MB-231 presented higher levels (FIG. 7A), which is in conflict with previous results. However, by flow cytometry it was observed that the most metastatic cell line LM2 expressed higher levels of IL13Rα2 in the membrane than the parental cell line 231, which could explain the discrepancy observed (FIG. 7B). Then, we investigated the effects of mAb 5.5.4 on mouse survival to lung metastasis. 5×10⁴ MDA-MB-231-LM2 cells were inoculated into the tail vein of NSG mice (n=12) and treated with mAb 5.5.4 following conditions already described. Survival curves showed a significant increase (p=0.016324) in survival of mice that were treated with Mab 6.6.1 compared to the control group (FIG. 7C). Although ultimately treated mice also developed lung metastases, the number of metastatic nodules was reduced by approximately 40% (FIG. 7D).

These results indicate that Mab 5.5.4 increases survival against lung metastasis and may be of therapeutic interest for triple negative breast cancer.

### III—Conclusion

In the present invention, a panel of mAbs specific for the IL-13 binding site in IL13Rα2, using the D1 sequence GSETWKTIITKN (SEQ ID NO: 2) as antigen (Bartolome R A et al. Br J Cancer 2018; 119:940-9), has been developed. Antibodies were selected according to their capacity for invasion inhibition. Functional analyses confirmed that mAbs 4.4.2 and 5.5.4 blocked the binding of IL-13 to IL13Rα2 and inhibited IL-13 pro-tumorigenic properties, including migration and invasion. Antibody effects were mediated through the inhibition of FAK, SRC, AKT and other IL13Rα2 downstream signaling mediators' activation. However, mAbs did not affect STAT6 signaling, IL13Rα1-mediated, indicating a blocking activity that was IL13Rα2-specific. The high selectivity for activated-only IL13Rα2 might turn out to be beneficial, avoiding indiscriminate targeting and minimizing negative collateral effects. Mab 5.5.4 also played a dual effect on receptor internalization and degradation as antagonist and agonist. Finally, direct evidence that treatment with mAb 5.5.4 caused a complete inhibition of liver metastasis in established and non-established liver metastasis is herein provided. No side effects on weight or overall health status were observed in the treated mice. Taken together, these observations indicate a strong potential therapeutic value for mAb 5.5.4.

The results showed here demonstrate the capacity of 5.5.4 to neutralize IL13Rα2-positive metastasis coming from differentiated cell lines. Both mAbs (4.4.2 and 5.5.4) inhibited FAK, SRC and AKT activation in KM12SM and SW620 cell lines. However, we observed some differences in signaling kinetics after ligand activation according to the IL13Rα2 expression level. Higher expression correlates with a faster activation. We also notice that both mAbs were able to inhibit cell proliferation independently from the presence of KRAS mutations, as occurs in SW620 cells. This result is stimulating as many colorectal cancers contain KRAS mutations, which render ineffective other targeted therapies such as those based on EGFR-treatments. Mabs were specific for blocking only IL13Rα2 activation in positive tumors, excluding collateral effects on IL13Rα1-mediated STAT6 activation. Interestingly, this high selectivity might avoid interferences with IL-4/IL-13 physiological effects through IL-4Ra/IL13Rα1. These results also confirm that the pro-metastatic activity of IL-13 is completely IL13Rα2-dependent, as IL13Rα1-mediated signaling was still active after metastasis inhibition. Mab 5.5.4 appears to be more effective than 4.4.2 in blocking IL-13-promoted IL13Rα2 internalization and recycling. Therefore, 5.5.4 seems to present a combination of direct antagonistic effect, blocking the receptor activation, with a simultaneous antibody-promoted receptor internalization and degradation (agonist effect). Endocytic trafficking appears to enable specific signaling pathways from intracellular sites and might play a critical role not only in attenuating IL13Rα2 signaling but also in controlling specific pathways (Vieira A V, et al. Science 1996; 274:2086-9).

In summary, we have obtained an IL13Rα2-specific antibody 5.5.4 that can inhibit IL-13-mediated FAK, Src and AKT signaling to suppress metastatic liver and lung colonization. The complete mouse survival indicates a potential therapeutic effect in metastasis from colorectal and breast cancer, including established and differentiated metastasis. Given the significant expression of IL13Rα2 in other late-stage human tumors (ovarian, pancreatic, glioblastoma), we believe that 5.5.4 mAbs might be clinically useful for these other human tumors.

1-20. (canceled)

21. A monoclonal antibody that specifically binds to an epitope comprising the amino acid sequence SEQ ID NO: 2 (GSETWKTIITKN), SEQ ID NO: 3 (IGSETWKTIITKNLHYKD), SEQ ID NO: 4 (GSETWKTIITKNC) or SEQ ID NO: 5 (GGGSETWKTIITKN) and capable of inhibiting the IL-13/IL13Rα2-mediated signaling or an antigen-binding fragment of said antibody, wherein the monoclonal antibody is selected from the group consisting of
a) an antibody comprising
the three heavy-chain CDRs of sequences SEQ ID NO: 6, SEQ ID NO: 7 and SEQ ID NO: 8 and
the three light-chain CDRs of sequences SEQ ID NO: 9, SEQ ID NO: 10 and SEQ ID NO: 11; and
b) an antibody comprising
the three heavy-chain CDRs of sequences SEQ ID NO: 12, SEQ ID NO: 13 and SEQ ID NO: 14, and
the three light-chain CDRs of sequences SEQ ID NO: 15, SEQ ID NO: 16 and SEQ ID NO: 17.

22. The monoclonal antibody or antigen binding-fragment according to claim 21, comprising
the three heavy-chain CDRs of sequences SEQ ID NO: 6, SEQ ID NO: 7 and SEQ ID NO: 8 and
the three light-chain CDRs of sequences SEQ ID NO: 9, SEQ ID NO: 10 and SEQ ID NO: 11.

23. An antiserum comprising the monoclonal antibody or antigen-binding fragment according to claim 21.

24. A cell expressing the monoclonal antibody or antigen-binding fragment according to claim 21.

25. A pharmaceutical composition comprising the monoclonal antibody or antigen-binding fragment according to claim 21.

26. A kit and/or device comprising the antibody or antigen-binding fragment according to claim 21.

27. A method for treating cancer comprising administering to a subject a therapeutically effective amount of the monoclonal antibody or antigen-binding fragment according to claim 21, wherein the cancer is characterized by expressing IL13Rα2.

28. The method according to claim 27, wherein the cancer is cancer metastasis.

29. The method according to claim 27, wherein the cancer is colon cancer, colorectal cancer, liver cancer, ovarian cancer, pancreatic cancer or glioblastoma.

30. The method according to claim 29, wherein the cancer is colorectal cancer.

31. The method according to claim 28, wherein the cancer metastasis is liver cancer metastasis.

32. The method according to claim 30, wherein the monoclonal antibody or antigen-binding fragment comprises
the three heavy-chain CDRs of sequences SEQ ID NO: 6, SEQ ID NO: 7 and SEQ ID NO: 8 and
the three light-chain CDRs of sequences SEQ ID NO: 9, SEQ ID NO: 10 and SEQ ID NO: 11.

33. A method for the prevention of metastatic progression in a subject suffering from a cancer comprising administering to said subject a therapeutically effective amount of the monoclonal antibody or antigen-binding fragment according to claim 21, wherein the cancer is characterized by expressing IL13Rα2.

34. The method according to claim 33, wherein the cancer is colon cancer, colorectal cancer, liver cancer, ovarian cancer, pancreatic cancer or glioblastoma.

35. The method according to claim 34, wherein the cancer is colorectal cancer.

36. The method according to claim 35, wherein the monoclonal antibody or antigen-binding fragment comprises
the three heavy-chain CDRs of sequences SEQ ID NO: 6, SEQ ID NO: 7 and SEQ ID NO: 8 and
the three light-chain CDRs of sequences SEQ ID NO: 9, SEQ ID NO: 10 and SEQ ID NO: 11.

37. The method according to claim 27, wherein the subject is a human being.

38. The method according to claim 33, wherein the subject is human being.

39. An in vitro method for designing a personalised therapy for a subject suffering from cancer comprising detecting the expression of IL13Rα2 in a cancer cell isolated from a biological sample of the subject, wherein if the expression of IL13Rα2 is detected, then the therapy to be administered to the subject is the monoclonal antibody according to claim 21.

40. An in vitro method according to claim 39, wherein the cancer is metastatic cancer.
